(19) United States
(12) Patent Application Publication
(10) Pub. No.: US 2005/0002999 A1
(43) Pub. Date: Jan. 6, 2005

(54) Long-circulating liposomal compositions

(76) Inventors: Rahul Mehta, San Marcos, CA (US); Gregory Hardee, Rancho Santa Fe, CA (US); Christopher P. Leamon, West Lafayette, IN (US)

Correspondence Address: COZEN O'CONNOR, P.C., 1900 MARKET STREET, PHILADELPHIA, PA, 19103-3508, US

(21) Appl. No.: 10/861,983

(22) Filed: Jun. 4, 2004

Related U.S. Application Data
(60) Provisional application No. 60/476,037, filed on Jun. 4, 2003.

(51) Int. Cl. A61K 9/127

(52) U.S. Cl. 424/450

(57) ABSTRACT

A liposome comprising about 1-25 mol % of a phosphatidylglycerol component and about 38-62 mol % of a phosphatidylcholine component is provided herein. In some embodiments, the liposomes further comprise a sterol component, for example a cholesterol.

### CROSS REFERENCE TO RELATED APPLICATION

This application claims benefit of priority of U.S. Provisional Application Ser. No. 60/476,037, filed Jun. 4, 2003, which is incorporated herein by reference.

### FIELD OF THE INVENTION

The present invention relates to long-circulating liposomal compositions within which one or more therapeutic agents are encapsulated. In preferred embodiments, such therapeutic agents include antisense compounds or other bioactive oligomers. When administered parenterally, therapeutic agents encapsulated with the liposomal compositions of the invention exhibit the desirable properties of increased stability within mammalian circulatory systems and altered tissue distribution relative to unformulated therapeutic agents. In preferred embodiments, the invention provides compositions and methods for the enhanced delivery of the encapsulated agent(s) to tumors and sites of inflammation. The present invention also encompasses methods of using the liposomal compositions of the invention in methods for the prophylactic, palliative and therapeutic treatment of hyperproliferative and inflammatory disorders.

### BACKGROUND OF THE INVENTION

Liposomes are microscopic spheres having an aqueous core surrounded and bounded by one (or more) outer shell(s), wherein each shell consists of lipid bilayers. Although the lipid bilayers may be made up of one type of lipid, it is more often the case that several chemically distinct lipids are present in the lipid bilayers. Chemical compounds, including bioactive agents (drugs) may be “encapsulated” in a liposome, i.e., passively or actively placed within the aqueous cores of liposomes during or after their formation, a process that is generally referred to in the art as “drug loading.”

The potential therapeutic use of liposomes as drug delivery agents was recognized nearly thirty years ago (Sessa et al., J. Lipid Res., 1968, 9, 310). There are, however, an immense number of lipids that are amenable for incorporation into liposomes, and, as is known in the art, changing the types of lipids present in liposomes changes the liposomes' pharmacological properties, sometimes with desirable results. Moreover, in some instances, changes in the pharmacological properties of liposomes can be realized by altering the proportions, as well as the types, of the lipids of which the liposomes' lipid bilayers are composed.

Research into pharmacologically optimal liposomal compositions has continued and another means of altering the pharmacological properties of liposomes has emerged, specifically, chemical modification of one or more lipids of a liposome by, e.g., attachment of a chemical moiety having one or more chemical or biological functions. Such functions include enhancing the stability of the liposomes in vitro or in vivo, making the liposomes resistant to uptake by cells of the reticuloendothelial system (RES), selectively targeting the liposomes to a specific cell type (such as, e.g., cancer cells) and/or other desirable functions. Chemical moieties that are attached to liposomes include, for example, polypeptides, such as antibodies (or biologically active fragments thereof) and receptor-binding proteins (e.g., transferrin and asialofetuin); glycolipids, e.g., galactose and mannose; and vitamins, such as folic acid (for a review of progress in the art of liposomes, see Chonn et al., Current Op. Biotech., 1995, 6, 698, 1995).

In the case of antisense compounds and other bioactive oligomers, it has been recognized that such compounds have great therapeutic potential, and several such compounds are undergoing clinical trials in humans (Roush, Science, 1997, 276, 1192; Rawls, Chemical & Engineering News, 1997, 75, 35; Winter, Consumer's Digest, issue of March/April, 1998). Although antisense compounds are currently being used in clinical trials for various therapeutic uses, there remains a need, as with many other types of drugs currently in use, for new pharmaceutical compositions and methods that can alter the in vivo stability, concentration and/or biodistribution of such compounds. Enhanced biostability of bioactive agents in a mammal coupled with improved delivery of such compounds to their intended target tissue(s) with potentially less frequent dosing represents an ongoing need in the pharmaceutical arts. With regard to altered distribution, for compounds active against oncogenic molecules, enhanced distribution to tumor tissues are desired and, for anti-inflammatory compounds, enhanced distribution to sites of inflammation are preferred.

Although various liposomal compositions are known, there remains a need for liposomal compositions that are optimized for bioactive agents (drugs), for enhancing the in vivo stability of such agents, and for avoiding of uptake by cells of the RES of such agents. There is also a need for delivery agents that selectively target tumors and sites of inflammation for more effective treatment of a variety of cancers and inflammatory diseases with a minimum of toxicity to other cells, thus sparing an animal to which a cytotoxic anticancer or anti-inflammatory agent is being administered from undesirable side-effects.

### RELATED ART

U.S. Pat. Nos. 5,077,057 (issued Dec. 31, 1991), 5,277,914 (issued Jan. 11, 1994), 5,549,910 (issued Aug. 27, 1996) and 5,567,434 (issued Oct. 22, 1996), all to Szoka, disclose methods of making liposomal compositions.

U.S. Pat. No. 5,100,662 (issued Mar. 31, 1992) to Bolcsak et al. discloses liposomal compositions.

U.S. Pat. No. 5,264,221 to Tagawa et al. (issued Nov. 23, 1993) discloses protein-bonded liposomes and asserts that the contents of such liposomes may include an antisense RNA.

U.S. Pat. No. 5,665,710 to Rahman et al. (Issued Sep. 9, 1997) describes certain methods of encapsulating oligodeoxynucleotides in liposomes.

Published PCT patent application WO 96/40062 to Thierry et al. discloses methods for encapsulating high molecular weight nucleic acids in liposomes.

Published PCT application WO 97/04787 to Love et al. discloses liposomes comprising antisense oligonucleotides targeted to the raf gene.

Published PCT application WO 97/46671 to Klimuk et al. discloses liposomal compositions comprising antisense oligonucleotides.

### SUMMARY OF THE INVENTION

In accordance with the present invention, novel compositions of long circulating liposomes capable of encapsulating a bioactive agent, and pharmaceutical compositions comprising such liposomes, are provided. The present invention is partly based on the surprising discovery that a liposome comprising a particular range of a phosphatidylglycerol component and phosphatidylcholine component have an enhanced stability inside a mammal, for example in the circulatory system of the mammal. In some embodiments, the enhanced stability of the liposomes allow for the liposomes to have a longer half-life inside the mammal, for example a longer half-life in the circulatory system of the mammal.

As used herein, a phosphatidylglycerine component comprises one or more phosphatidylglycerine, and a phosphatidylcholine component comprises one or more phosphatidylcholine. In some embodiments, a phosphatidylglycerol and a phosphatidylcholine of the present invention comprise a first fatty acid and a second fatty acid. A fatty acid is a carboxylic acid that contains a long, hydrophobic hydrocarbon chain. In some embodiments, the hydrocarbon chain of the first and/or second fatty acids of the phosphatidylglycerol comprises about 10-20 carbons. In some embodiments, the first and second fatty acids of the phosphatidylglycerol comprise the same number of carbons. In some embodiments, the first and second fatty acid of the phosphatidylglycerol comprises a different number of carbons. In some embodiments, the first and second fatty acids of the phosphatidylcholine comprise the same number of carbons. In some embodiments, the first and second fatty acid of the phosphatidylcholine comprises a different number of carbons.

In some embodiments, the liposomes of the present invention comprise about 0.5-25 mol % of a phosphatidylglycerol component, and about 38-62 mol % of a phosphatidylcholine component. In some embodiments, the liposomes comprise about 5 mol % of a phosphatidylglycerol component (e.g., a dimyristoylphosphatidylglycerol) and about 57 mol % of a phosphatidylcholine component (e.g., dipalmitoylphosphatidylcholine). In some embodiments, the liposomes of the present invention further comprise a sterol component, which comprises one or more sterol, such as a cholesterol. For example, a liposome of the present invention may comprise about 5 mol % of a phosphatidylglycerol component (e.g., a dimyristoylphosphatidylglycerol), about 57 mol % of a phosphatidylcholine component (e.g., dipalmitoylphosphatidylcholine), and about 38 mol % of a sterol component (e.g., cholesterol).

In particular, bioactive agents having anticancer or anti-inflammatory effects are encapsulated within the liposomes of the invention, as the liposomal compositions of the invention selectively target delivery of the encapsulated bioactive agents to tumors and sites of inflammation. However, the liposomal compositions of the invention also provide for enhanced stability in the circulatory system of an animal for a variety of bioactive agents, regardless of their biological activity, and are thus termed “long circulating” liposomes.

In one embodiment of the invention, the bioactive agent encapsulated within such long circulating liposomes is an antisense compound (including, for example, an antisense oligonucleotide, an antisense PNA, a ribozyme or EGS sequence) or another type of bioactive oligomer (e.g., an aptamer). Alternatively, the bioactive agent is an agent other than an oligonucleotide, such as cisplatin.

In some embodiments, the bioactive agent encapsulated with liposomes of the present invention includes oligomeric compounds, particularly single and double-stranded antisense compounds, which are targeted to a nucleic acid encoding certain proteins, and which modulate the expression of that protein. In some embodiments the antisense compounds are oligonucleotides. In some embodiments, the oligonucleotides are RNAi oligonucleotides (which are predominantly RNA or RNA-like). In other embodiments, the oligonucleotides are RNase H oligonucleotides (which are predominantly DNA or DNA-like). In still other embodiments, the oligonucleotides may be chemically modified.

Also provided are liposome-based pharmaceutical compositions which inhibit the hyperproliferation of cells, particularly cancerous cells, and/or their metastasis. Further provided are methods of preventing or inhibiting the hyperproliferation of cells, particularly cancerous cells, and/or their metastasis.

The invention also provides liposome-based pharmaceutical compositions which inhibit inflammation and/or biological injury resulting from inflammation. Further provided are methods of preventing or inhibiting inflammation and/or biological injury resulting from inflammation.

Further provided are anticancer and anti-inflammatory combination therapies, and pharmaceutical compositions useful in such therapies. Such combination therapies involve, for example, administration of a first liposomal composition encapsulating a bioactive oligomer combined with the administration of a second liposomal composition encapsulating a bioactive agent other than an oligonucleotide, wherein both bioactive agents have anticancer or anti-inflammatory activity.

### BRIEF DESCRIPTION OF THE DRAWINGS

FIG. 1 shows the clearance from plasma of ISIS 2503-encapsulating liposomes comprising 1 mol % (diamonds), 10 mol % (squares), 15 mol % (triangles) or 20 mol % (circles) DMPG.

FIG. 2 shows the clearance from plasma of liposomes comprising 5 mol % of DLPG (diamonds), DPPG (squares), DMPG (triangles) or DSPG (circles).

FIG. 3 shows the clearance from plasma of ISIS 2503-formulated in phosphate-buffered saline (PBS, diamonds) or in liposomes comprising 5 mol % DPPS (squares), 5 mol % DPGS (triangles) or 5 mol % DMPG (solid line).

FIG. 4 shows the plasma concentration of ISIS 2105 in mice having H69-derived xenografts after dosing of the oligonucleotide formulated in PBS or the in indicated liposomes (CL, cardiolipin; DPPS, DMPG, GM1 and PEG are described in detail in Example 1).

FIG. 5 shows the tumor concentration of ISIS 2105 in mice having H69-derived xenografts after dosing of the oligonucleotide formulated in PBS or the in indicated liposomes (i.e., CL, cardiolipin; or DPPS, DMPG, GM1 or PEG).

FIG. 6 shows the plasma concentration of ISIS 2105 in mice having MIA PaCa-derived xenografts after dosing of the oligonucleotide formulated in PBS or the indicated liposomes (i.e., DMPG, GM1 or PEG).

FIG. 7 shows the tumor concentration of ISIS 2105 in mice having MIA PaCa-derived xenografts after dosing of the oligonucleotide formulated in PBS or the indicated liposomes (i.e., DMPG, GM1 or PEG).

### DESCRIPTION OF THE EMBODIMENTS

I. Liposomal Compositions

Liposomes are microscopic spheres having an aqueous core surrounded by one or more outer layer(s) made up of lipids arranged in a bilayer configuration (see, generally, Chonn et al., Current Op. Biotech., 1995, 6, 698, 1995). The therapeutic potential of liposomes as drug delivery agents was recognized nearly thirty years ago (Sessa et al., J. Lipid Res., 1968, 9, 310). Liposomes may, in some instances, be used as cellular delivery vehicles for bioactive agents in vitro and in vivo (Mannino et al., Biotechniques, 1988, 6, 682; Blume et al., Biochem. et Biophys. Acta, 1990, 1029, 91; Lappalainen et al., Antiviral Res., 1994, 23, 119. For example, it has been shown that large unilamellar vesicles (LUV), which range in size from 0.2-0.4 microns, can encapsulate a substantial percentage of an aqueous buffer containing large macromolecules. RNA, DNA and intact virions can be encapsulated within the aqueous interior and delivered to brain cells in a biologically active form (Fraley et al., Trends Biochem. Sci., 1981, 6, 77).

Liposomes include “long circulating liposomes” (a.k.a. “sterically stabilized liposomes”) a term which, as used herein, refers to liposomes comprising one or more specialized lipids that, when incorporated into liposomes, result in enhanced circulation lifetimes relative to liposomes lacking such specialized lipids. Examples of long circulating liposomes known in the art include those in which the liposome (A) comprises one or more glycolipids such as monosialoganglioside G_{M1} or (B) comprises one or more lipids derivatized with one or more hydrophilic polymers, such as a polyethylene glycol (PEG) moiety. While not wishing to be bound by any theory, at least for long circulating liposomes containing gangliosides, sphingomyelin, or PEG-derivatized lipids, the enhanced circulation half-life of these liposomes derives from a reduced uptake into cells of the reticuloendothelial system (RES) (Allen et al., FEBS Letters, 1987, 223, 42; Wu et al., Cancer Research, 1993, 53, 3765).

A. Glycolipid-Comprising Liposomes: Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. USA, 1988, 85, 6949). U.S. Pat. No. 4,837,028 and published PCT application WO 88/04924, both to Allen et al., disclose liposomes comprising (1) sphingomyelin and (2) the ganglioside G_{M1} or a galactocerebroside sulfate ester. U.S. Pat. No. 5,543,152 (to Webb et al.) discloses liposomes comprising sphingomyelin. Liposomes comprising 1,2-sn-dimyristoylphosphatidylcholine are disclosed in published PCT application WO 97/13499 (to Lim et al.).

B. Liposomes Derivatized with Hydrophilic Polymers: Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, that contains a PEG moiety. Illum et al. (FEBS Letters, 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) and liposomes comprising such phospholipids are described by Sears (U.S. Pat. Nos. 4,426,330 and 4,534,899). Klibanov et al. (FEBS Letts., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the chemical attachment of PEG to DSPE (distearoylphosphatidylethanolamine).

Liposomes having covalently bound PEG moieties on their external surface are described in European Patent No. 0 445 131 B1 and published PCT application WO 90/04384 to Fisher. Liposome compositions containing 1-20 mole percent (mol %) of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in published PCT application WO 91/05545 and U.S. Pat. No. 5,225,212 (both to Martin et al.) and in published PCT application WO 94/20073 (Zalipsky et al.) Liposomes comprising PEG-modified ceramide lipids are described in published PCT application WO 96/10391 (Choi et al.). U.S. Pat. Nos. 5,540,935 (Miyazaki et al.) and 5,556,948 (Tagawa et al.) describe PEG-containing liposomes that can be further derivatized on their surfaces with functional moieties.

C. DMPG-Containing Liposomes: Various liposomes comprising dimyristoylphosphatidylglycerol (DMPG) have been described. Generally, however, such liposomes comprise DMPG in a mol % of about 10% or higher (see, for example, Akhtar et al. (Nucl. Acids Res., 1991, 19, 5551; Yachi et al. (Biopharm. Drug Dispos., 1996, 17, 699; and Farmer et al. (Meth. Enz., 1987, 149, 184). Liposomes having 3 mol % DMPG have been described, but such liposomes included a component (in particular, a phosphatidylcholine derivative) that is not found in the liposomal compositions of the present invention. Such phosphatidylcholine derivative components include, e.g., 10 mol % distearoylphosphatidylcholine (DSPC) (Brodt et al., Cancer Immunol. Immunother., 1989, 28, 54) or 7 mol % dimyristoylphosphatidylcholine (DMPC) (Perez-Soler et al., J. Nuclear Med., 1985, 26, 743; Wasan et al., Antimicrobial Agents and Chemotherapy, 1993, 37, 246; and Li et al., Oncology Res., 1995, 7, 611).

In some embodiments, the liposomes of the present invention effectively exclude any detectable amount of DSPC or DMPC, because such phosphatidylcholine derivatives may possess undesirable properties.

II. Liposomes of the Invention

In some embodiments, the liposomes of the present invention comprise about 1-25 mol % of a phosphatidylglycerol component, and about 38-62 mol % of a phosphatidylcholine component. In some embodiments, the liposomes of the present invention comprise about 15-25 mol % of a phosphatidylglycerol component and about 38-47 mol % of a phosphatidylcholine component, for example, about 20 and 42 mol % respectively. In some embodiments, the liposomes comprise about 5-15 mol % of the phosphatidylglycerol component and about 47-57 mol % of the phosphatidylcholine component, for example, about 10 and 52 mol %, respectively. In some embodiments, the liposomes comprise about 3-10 mol % of the phosphatidylglycerol component and about 52-60 mol % of the phosphatidylcholine component, for example, about 5 and 57 mol %, respectively. In some embodiments, the liposomes comprise about 1 mol % of the phosphatidylglycerol component and about 59-62 mol % of the phosphatidylcholine component, for example, about 1 and 62 mol %, respectively.

In some embodiments, the liposomes of the present invention have an enhanced stability inside a mammal, for example in the circulatory system of the mammal. In some embodiments, the enhanced stability of the liposomes allow for the liposomes to have a longer half-life inside the mammal, for example a longer half-life in the circulatory system of the mammal. For example, in some embodiments, the liposomes of the present invention have a half-life of at least 5 hours in the blood circulatory system. In some embodiments, the liposomes of the present invention have a half-life of at least 10 hours in the blood circulatory system. In some embodiments, the liposomes of the present invention have a half-life of at least 14 hours in the blood circulatory system. In some embodiments, the liposomes of the present invention have a half-life of at least 16 hours in the blood circulatory system.

In some embodiments, liposomes of the present invention that comprise about 1-25 mol % of the phosphatidylglycerol component and about 38-62 mol % of the phosphatidylcholine component have a half-life of at least 10 hours in the circulatory system of a mammal. In some embodiments, liposomes of the present invention that comprise about 1-25 mol % of the phosphatidylglycerol component and about 38-62 mol % of the phosphatidylcholine component have a half-life of at least 14 hours in the circulatory system of a mammal.

In some embodiments, liposomes of the present invention that comprise about 5-15 mol % of the phosphatidylglycerol component and about 47-57 mol % of the phosphatidylcholine component, for example, about 10 and 52 mol %, respectively, have a half-life of at least 10 hours in the circulatory system of a mammal. In some embodiments, liposomes of the present invention that comprise about 5-15 mol % of the phosphatidylglycerol component and about 47-57 mol % of the phosphatidylcholine component, for example, about 10 and 52 mol %, respectively, have a half-life of at least 14 hours in the circulatory system of a mammal.

A phosphatidate is customarily linked to two fatty acids. Accordingly, the phosphatidylglycerol component of the present invention comprises a phosphatidylglycerol which has a first fatty acid and a second fatty acid. In some embodiments, the hydrocarbon chain the first fatty acid comprises about 10-20 carbons. In some embodiments, the hydrocarbon chain the first fatty acid comprises about 14-20 carbons. In some embodiments, the hydrocarbon chain the first fatty acid comprises about 16-18 carbons. In some embodiments, the hydrocarbon chain the first fatty acid comprises about 10-20 carbons. In some embodiments, the hydrocarbon chain the second fatty acid comprises about 14-20 carbons. In some embodiments, the hydrocarbon chain the second fatty acid comprises about 16-18 carbons. In some embodiments, the hydrocarbon chain of the first and/or second fatty acids is/are partially or entirely saturated. In some embodiments, the hydrocarbon chain of the first and/or second fatty acids is/are partially or entirely unsaturated.

In some embodiments, the hydrocarbon chain of the first fatty acid of the phosphatidylglycerol may comprise a different number of carbons than a hydrocarbon chain of the second fatty acid. For example, the first hydrocarbon chain may comprise about 10 carbons and the second hydrocarbon chain may comprise about 15 carbons.

In some embodiments, the hydrocarbon chain of the first fatty acid of the phosphatidylglycerol may comprise the same number of carbons as the hydrocarbon chain of the second fatty acid. For example, a phosphatidylglycerol of the present invention may be a dimyristoylphosphatidylglycerol, wherein both the first and second hydrocarbon chains thereof have 14 carbons. Other non-limiting examples of phosphatidylglycerols that have the same number of carbons in the first and second hydrocarbon chains include: a dilauroylphosphatidylglycerol, a dipalmitoylphosphatidylglycerol, a disteroylphosphatidylglycerol.

In some embodiments, the phosphatidylglycerol component comprises a phosphatidylglycerol-derived compound. In some embodiments, phosphatidylglycerol-derived compounds include a phosphatidylglycerol wherein the phosphoglycerol head group is replaced with another polar head group. An example of such compound include dipalmitoylglycerosuccinate (a glycolipid), wherein a succinate moiety is present in lieu of the phosphoglycerol moiety of the phosphatidylglycerol. In other such phosphatidylglycerol-derived compounds, a zwitterionic head group is employed, such as in phosphatidylserine and dipalmitoylphosphatidylserine.

In some embodiments, the phosphatidylcholine component of the present invention comprises a phosphatidylcholine which has a first fatty acid and a second fatty acid. In some embodiments, the hydrocarbon chain the first fatty acid comprises about 10-20 carbons. In some embodiments, the hydrocarbon chain the first fatty acid comprises about 14-20 carbons. In some embodiments, the hydrocarbon chain the first fatty acid comprises about 16-18 carbons. In some embodiments, the hydrocarbon chain the first fatty acid comprises about 10-20 carbons. In some embodiments, the hydrocarbon chain the second fatty acid comprises about 14-20 carbons. In some embodiments, the hydrocarbon chain the second fatty acid comprises about 16-18 carbons. In some embodiments, the hydrocarbon chain of the first and/or second fatty acids is/are partially or entirely saturated. In some embodiments, the hydrocarbon chain of the first and/or second fatty acids is/are partially or entirely unsaturated.

In some embodiments, the hydrocarbon chain of the first fatty acid of the phosphatidylcholine may comprise a different number of carbons than a hydrocarbon chain of the second fatty acid. For example, the first hydrocarbon chain may comprise about 10 carbons and the second hydrocarbon chain may comprise about 15.

In some embodiments, the hydrocarbon chain of the first fatty acid of the phosphatidylcholine may comprise the same number of carbons as the hydrocarbon chain of the second fatty acid. For example, a phosphatidylcholine of the present invention may be a dipalmitoylphosphatidylcholine, wherein both the first and second hydrocarbon chains thereof have 16 carbons.

In some embodiments, the phosphatidylcholine component comprises an egg phosphatidylcholine, a soybean phosphatidylcholine, a dilauroylphosphatidylcholine, a dipalmitoylphosphatidylcholine or a mixture thereof.

In some embodiments, a phosphatidylethanolamine is used in place of a phosphatidylcholine.

In some embodiments, the liposomes of the present invention may further comprise a sterol component. A sterol component comprises one or more sterols, which are compounds containing a multi-ring (steroid) structure and a hydroxyl group (—OH). A non-limiting example of a sterol includes a cholesterol. In some embodiments, the liposomes comprises about 33-42 mol % of a sterol component. In some embodiments, the liposomes comprise about 38 mol % of a sterol component.

In some embodiments, a liposome of the present invention comprises a dimyristoylphosphatidylglycerol and a dipalmitoylphosphatidylcholine. In some embodiments, the liposome comprises about 5 mol % of a dimyristoylphosphatidylglycerol and about 57 mol % of a dipalmitoylphosphatidylcholine.

In some embodiments, a liposome of the present invention comprises a dimyristoylphosphatidylglycerol, a dipalmitoylphosphatidylcholine and a sterol component. In some embodiments, the liposome comprises about 5 mol % of a dimyristoylphosphatidylglycerol, about 57 mol % of a dipalmitoylphosphatidylcholine and about 38 mol % of a sterol component, for example 38 mol % of a cholesterol.

The liposomes of the present invention may also be used to encapsulate a bioactive agent (described below). Further, these liposomes may be employed in preventing or treating mammals. In some embodiments, a mammal of the present invention may be a human, a monkey, a rodent or a chicken. In some embodiments, the liposomes of the present invention may be employed in preventing cancer or reducing the rate of growth of a tumor or cancer in a mammal. In some embodiments, the liposomes of the present invention may also be employed in preventing or reducing the severity of inflammation in a mammal.

The liposomes of the invention are formed from vesicle-forming lipids which generally include one or more neutral or negatively charged phospholipids, preferably one or more neutral phospholipids, usually in combination with one or more sterols, particularly cholesterol. Examples of lipids generally useful in liposome production include phosphatidyl compounds, such as phosphatidylglycerol, phosphatidylcholine, phosphatidylserine, sphingolipids, phosphatidylethanolamine, cerebrosides and gangliosides.

Typically, the major lipid component of the liposomes is a phosphatidylcholine (PC) compound or PC derivative having general structure I: 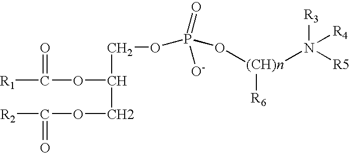
wherein R₁ and R₂ are, independently, hydrocarbon-based, substantially hydrophobic “tail” groups. R₃, R₄, R₅, R₆ are independently H or alkyl. n is an integer from 1-4. The choline which, together with the phosphate moiety, forms a substantially hydrophillic “head”. As used herein, the term “phosphatidylcholine compound” refers both to phosphatidylcholine isolated from natural sources, in which a variety of PC derivatives with a variety of head groups of varying chain length and degree of saturation may be present, as well as essentially pure synthetic PC derivatives which are commercially available or which may be synthesized by known techniques. For purposes of filter sterilization, less-saturated PC compounds are generally more easily sized, particularly when the liposomes must be sized below about 0.3 microns.

PC compounds containing saturated fatty acids with tail lengths in the range of C₁₄ to C₂₂, particularly C₁₆ to C₁₈, are preferred. Representative fatty acids include, for example, oleic acid, lauric acid, capric acid (n-decanoic acid), myristic acid, palmitic acid, stearic acid, linoleic acid, and linolenic acid.

Illustrative PC compounds include phosphatidylcholine and dipalmitoylphosphatidylcholine. Phosphatidylcholines with mono- and di-unsaturated fatty acids and mixtures of saturated and unsaturated fatty acids may also be used. Other suitable phospholipids include phosphatidyl compounds with head groups derived from alcohols other than choline, such as, for example, ethanolamine, serine, glycerol, and inositol. Liposomes may also include a sterol, e.g., cholesterol, at molar ratios of from about 0.1 to 1.0 (sterol:phospholipid).

In particular, the present invention provides for long circulating liposomes comprising a phosphatidylglycerol (PG) compound. The term “phosphatidylglycerol compound” (or “PG compound”) as used herein, encompasses a variety of compounds including, but not limited to, phospholipids (such as, e.g., DLPG, DPPG and DSPG). (see Example 1 for non-abbreviated descriptions of these and other lipids). The liposomes of the invention may further comprise a phosphatidylcholine (PC) compound. As defined herein, the term “phosphatidylcholine compound” (or “PC compound”) includes phosphatidylcholine per se as well as derivatives thereof (e.g., DPPC and DLPC) and heterogenous mixtures of phosphatidylcholine molecules isolated from natural sources (e.g., soy PC and egg PC). The liposomes of the invention may further comprise a sterol, such as cholesterol.

As detailed herein, it has been surprisingly been discovered that the proportion of DMPG present in certain liposomal compositions can be decreased to as little as about 1 mol % with no loss of their desirable properties, e.g., long circulating times, altered biodistribution, etc.; indeed, if anything, such desirable properties are surprisingly enhanced when lower proportions of DMPG are used.

The disclosure provides for novel long circulating liposomes that comprise (1) less than 10 mol %, particularly from about 1 mol % to about 9 mol %, preferably from about 1 mol % to about 7 mol %, more preferably from about 1 mol % to about 5 mol %, and even more preferably about 1 mol % to 2.9 mol % of DMPG or another phosphatidylglycerol compound, and (2) functionally ineffective amounts of the undesirable lipid components DSPC and DMPC. Liposomes of the invention generally comprise less than about 25 mol % of these undesirable components, more preferably less than about 15 mol %, even more preferably less than about 5 mol %, and still more preferably less than about 1 mol %. The long circulating liposomes of the invention should have a half-life in mammalian plasma of preferably greater than about 9 hours, more preferably greater than about 12 hours, and most preferably greater than about 14 hours. Other long circulating liposomes having related compositions to the DMPG-comprising liposomes of the invention, but comprising derivatives of DMPG of varying chain lengths in lieu of DMPG, are also described herein.

Preferred long circulating liposomal compositions of the invention include those having about 5 mol % PG, about 57 mol % PC and about 38 mol % sterol, wherein “PG” indicates a phosphatidylglycerol compound and “PC” indicates a phosphatidylcholine compound. A preferred phosphatidylglycerol compound for use in the liposomes of the invention is DMPG, and a preferred sterol for use in the liposomes of the invention is cholesterol (Chol).

A preferred phosphatidylcholine compound for use in the liposomes of the invention is dipalmitoylphosphatidylcholine (DPPC), but other phosphatidylcholine compounds (other than DSPC and DMPC) may be used in the alternative. Other PC compounds that may be used to create long circulating liposomes according to the disclosure include soy PC, egg PC and dilauroylphosphatidylcholine (DLPC), for example.

In other embodiments of the invention, the proportion of the PG compound in the liposomes of the invention is adjusted to be from about 1 mol % to about 9 mol %; in such liposomes, the mol % of sterol is held constant (i.e., at about 38 mol %), and the mol % of the PC compound is adjusted to reflect the change in concentration of the PG compound. More specifically, these proportions are as follows:

| mol % PG | mol % PC | mol % |
|---|---|---|
| compound | compound | sterol |
| ˜1 | ˜61 | ˜38 |
| ˜5 | ˜57 | ˜38 |
| ˜10 | ˜52 | ˜38 |

Other embodiments of the invention include long circulating liposomes in which the myristic acid portions of DMPG is substituted by another long chain fatty acid. In these liposomes, DMPG is replaced by, e.g., DLPG, DPPG or DSPG.

In another set of embodiments of the invention, the phosphatidylglycerol compound is modified, i.e., a PG-derived compound is used in the liposomes in lieu of a PG compound. In some such PG-derived compounds, the phosphoglycerol head group is replaced with another polar head group; for example, in the case of the glycolipid dipalmitoylglycerosuccinate (DPGS), a succinate moiety is present in lieu of the phosphoglycerol moiety. In other such PG-derived compounds, a zwitterionic head group is employed, such as in dipalmitoylphosphatidylserine (DPPS).

The liposomes of the invention can be prepared by any of a variety of known techniques. For example, the liposomes can be formed by any conventional technique for preparing multilamellar lipid vesicles (MLVs), i.e., by depositing one or more selected lipids on the inside wall of a suitable vessel by dissolving the lipid in chloroform, evaporating the chloroform and then adding an aqueous solution which comprises the agent(s) to be encapsulated to the vessel, allowing the aqueous solution to hydrate the lipid, and swirling or vortexing the resulting lipid suspension. This process yields a mixture including the desired liposomes.

As another example, techniques used for producing large unilamellar vesicles (LUVs), such as, e.g., reverse-phase evaporation, infusion procedures and detergent dilution, can be used to produce the liposomes. These and other methods for producing lipid vesicles are described in Liposome Technology, Volume I (Gregoriadis, Ed., CRC Press, Boca Raton, Fla., 1984). The liposomes can be in the form of steroidal lipid vesicles, stable plurilamellar vesicles (SPLVs), monophasic vesicles (MPVs) or lipid matrix carriers (LMCs) of the type disclosed in U.S. Pat. Nos. 4,588,578 and 4,610,868 (both to Fountain et al.), 4,522,803 (to Lenk et al.), and 5,008,050 (to Cullis et al.). In the case of MLVs, the liposomes can be subjected to multiple (five or more) freeze-thaw cycles to enhance their trapped volumes and trapping efficiencies and to provide a more uniform interlamellar distribution of solute if desired (Mayer et al., J. Biol. Chem., 1985, 260, 802). Specific methods for making particular oligodeoxynucleotide:liposome compositions are described in U.S. Pat. No. 5,665,710 to Rahman et al.

Following their preparation, liposomes may be sized to achieve a desired size range and relatively narrow distribution of sized particles. In preferred embodiments, the liposomes have a lower range of diameters of from about 50 to about 75 nM, most preferably about 60 nM, and an upper range of diameters from about 75 to about 150 nM, most preferably about 125 nM, where “about” indicates ±10 nM.

Several techniques are available for sizing liposomes to a desired size range. Sonicating a liposome suspension by either bath or probe sonication produces a progressive size reduction down to small unilamellar vesicles (SUVs) less than about 0.05 microns in size. Homogenization, which relies on shearing energy to fragment large liposomes into smaller ones, is another known sizing technique in which MLVs are recirculated through a standard emulsion homogenizer until a selected liposome size range, typically between about 0.1 and about 0.5 microns, is achieved. Extrusion of liposomes through a filter or membrane is another method for producing liposomes having a desired size range (see, for example, U.S. Pat. Nos. 4,737,323 to Martin et al. and 5,008,050 to Cullis et al.). Other useful sizing methods are known to those skilled in the art. In most such methods, the particle size distribution can be monitored by conventional laser-beam size determination or other means known in the art.

Liposomes may be dehydrated, preferably under reduced pressure using standard freeze-drying equipment, for extended storage. Whether dehydrated or not, the liposomes and their surrounding media can first be frozen in liquid nitrogen and placed under reduced pressure. Although the addition of the latter freezing step makes for a longer overall dehydration process, there is less damage to the lipid vesicles, and less loss of their internal contents, when the liposomes are frozen before dehydration.

To ensure that the a significant portion of the liposomes will endure the dehydration process intact, one or more protective sugars may be made available to interact with the lipid vesicle membranes and keep them intact as water is removed. Appropriate sugars include, but are not limited to, trehalose, maltose, sucrose, lactose, glucose, dextran and the like. In general, disaccharide sugars may work better than monosaccharide sugars, with trehalose and sucrose being particularly effective in most cases, but other, more complicated sugars may alternatively be used. The amount of sugar to be used depends on the type of sugar and the characteristics of the lipid vesicles. Persons skilled in the art can readily test various sugars and concentrations to determine what conditions work best for a particular lipid vesicle preparation (see, generally, Harrigan et al., Chem. Phys. Lipids, 1990, 52, 139, and U.S. Pat. No. 4,880,635 to Janoff et al.). Generally, sugar concentrations of greater than or equal to about 100 mM have been found to result in the desired degree of protection. Once the liposomes have been dehydrated, they can be stored for extended periods of time until they are to be used. The appropriate conditions for storage will depend on the chemical composition of the lipid vesicles and their encapsulated active agent(s). For example, liposomes comprising heat labile agents should be stored under refrigerated conditions so that the potency of the active agent is not lost.

The liposomes of the invention may be used to encapsulate a variety of bioactive agents for therapeutic purposes. Such bioactive agents are described generally, and in terms of specific examples, in the following section.

III. Bioactive Agents

In general, the term “Bioactive Agent” encompasses any agent that provides a prophylactic, palliative or therapeutic effect to an animal in need thereof. In particular embodiments of the invention, preferred Bioactive Agents include (A) Bioactive oligomers, (B) Anticancer Agents and (C) Anti-Inflammatory Agents. These compounds, as well as (D) Combinations of Bioactive Agents, are detailed infra. The invention is not limited to these examples, however; in general, any bioactive agent may be incorporated within the liposomal compositions of the invention for the purpose of achieving an enhanced stability in the circulatory system of an animal, including a human.

A. Bioactive oligomers include (1) antisense compounds and (2) other bioactive oligomers. These compounds are described in more detail, infra.

Antisense Compounds: As used herein, the term “antisense compound” encompasses, inter alia, single stranded antisense oligonucleotides (DNA, DNA-like, RNA, RNA-like) or double stranded or self-hybridizing constructs comprising an antisense orientation oligonucleotide, antisense PNAs, ribozymes and EGSs (described infra). Antisense compounds can exert their effect by a variety of means. One such means is the antisense-mediated direction of an endogenous nuclease, such as RNase H in eukaryotes or RNase P in prokaryotes, or dsRNAases in RNAi pathways to the target nucleic acid (Chiang et al., J. Biol. Chem., 1991, 266, 18162; Forster et al., Science, 1990, 249, 783). The sequences that recruit RNase P are known as External Guide Sequences, hence the abbreviation “EGSs” (Guerrier-Takada et al., Proc. Natl. Acad. Sci. USA, 1997, 94, 8468).

In many species, introduction of double-stranded RNA (dsRNA) induces potent and specific gene silencing. This posttranscriptional gene silencing phenomenon has been termed “RNA interference” (RNAi) and has come to generally refer to the process of gene silencing involving dsRNA which leads to the sequence-specific reduction of gene expression via target mRNA degradation (Tuschl et al., Genes Dev., 1999, 13, 3191-3197).

Another type of bioactive oligomer is an RNA-RNA hybrid molecule that can modulate gene expression. The double strand RNA may in some instances be described as siRNA. For the purposes of describing an embodiment of this invention, an siRNA is a combination of an antisense strand and a sense strand, each of a specified length sufficient to exhibit desirable properties such as a stability and target specificity, for example from about 8-30, about 12-27, about 17-25, or about 19-23 nucleotides long. Such a complementary pair of oligonucleotides can be blunt ended or can include additional nucleotides on either or both of their 5′ or 3′ ends. Further they can include other molecules or molecular structures on their 3′ or 5′ ends such as a phosphate group on the 5′ end. A preferred group of compounds of the invention include a phosphate group on the 5′ end of the antisense strand compound. Other preferred compounds also include a phosphate group on the 5′ end of the sense strand compound. An even further preferred compounds would include additional nucleotides such as a two base overhang on the 3′ end.

It has been demonstrated that 21- and 22-nt dsRNA fragments having 3′ overhangs are the canonical sequence-specific mediators of RNAi. These fragments, which are termed short interfering RNAs (siRNAs), are generated by an RNase III-like processing reaction from longer dsRNA. Chemically synthesized siRNA also mediate efficient target RNA cleavage with the site of cleavage located near the center of the region spanned by the guiding strand of the siRNA. (Elbashir et al., Nature, 2001, 411, 494-498). Characterization of the suppression of expression of endogenous and heterologous genes caused by the 21-23 nucleotide siRNAs has been investigated in several mammalian cell lines, including human embryonic kidney (293) and HeLa cells (Elbashir et al., Genes and Development, 2001, 15, 188-200).

Recently, it has been shown that single-stranded RNA oligomers (ssRNAi or asRNA) of antisense polarity can be potent inducers of gene silencing and that single-stranded oligomers are ultimately responsible for the RNAi phenomenon (Tijsterman et al., Science, 2002, 295, 694-697).

U.S. Pat. Nos. 5,898,031 and 6,107,094, each of which is herein incorporated by reference, describe certain oligonucleotides having RNA-like properties. When hybridized with RNA, these oligonucleotides serve as substrates for a dsRNase enzyme with resultant cleavage of the RNA by the enzyme (Crooke, 2000; Crooke, 1999). Further details of RNAi and RNAase H may be found in U.S. patent application filed on Jun. 3, 2004, Attorney Docket No. ISIS0134-100 (BIOL0042), the disclosure of which is incorporated in its entirety herein by reference.

Another means involves covalently linking a synthetic moiety having nuclease activity to an oligonucleotide having an antisense sequence, rather than relying upon recruitment of an endogenous nuclease. Synthetic moieties having nuclease activity include, but are not limited to, enzymatic RNAs, lanthanide ion complexes, and the like (Haseloff et al., Nature, 1988, 334, 585; Baker et al., J. Am. Chem. Soc., 1997, 119, 8749).

As used herein, the term “antisense compound” includes ribozymes, synthetic RNA molecules and derivatives thereof that catalyze highly specific endoribonuclease reactions (see, generally, U.S. Pat. No. 5,543,508 to Haseloff et al. and U.S. Pat. No. 5,545,729 to Goodchild et al.). The cleavage reactions are catalyzed by the RNA molecules themselves. In naturally occurring RNA catalytic molecules, the sites of self-catalyzed cleavage are located within highly conserved regions of RNA secondary structure (Buzayan et al., Proc. Natl. Acad. Sci. USA, 1986, 83, 8859; Forster et al., Cell, 1987, 50, 9). Naturally occurring autocatalytic RNA molecules have been modified to generate ribozymes which can be targeted to a particular cellular or pathogenic RNA molecule with a high degree of specificity. Thus, ribozymes serve the same general purpose as antisense oligonucleotides (i.e., modulation of expression of a specific gene). That is, ribozymes have substantial chemical and functional identity with other bioactive compounds and may thus be formulated for pharmaceutical delivery using the liposomes of the present invention.

The antisense compounds encapsulated within the liposomal compositions of the invention (1) may be from about 8 to about 100 nucleotides in length, more preferably from about 10 to about 30 nucleotides in length, (2) are targeted to a nucleic acid sequence required for the expression of a gene from a mammal, including a human, and (3), when contacted with cells expressing the target gene, modulate its expression. Due to the biological activity of the gene product encoded by the target gene, modulation of its expression has the desirable result of providing specific prophylactic, palliative and/or therapeutic effects.

Typically, the liposomes of the invention will contain, in their aqueous interiors, an antisense compound in an amount of from about 0.005 ng/mL to about 400 mg/mL, preferably from about 0.01 ng/mL to about 200 mg/mL, most preferably from about 0.1 ng/mL to about 100 mg/mL, where “about” indicates ±5% of the indicated concentration.

The term “antisense compound” specifically includes synthetic oligonucleotides, as well as peptide nucleic acids (PNAs), having a nucleobase sequence specifically hybridizable with a nucleic acid. In the context of this invention, the term “oligonucleotide” refers to an oligomer or polymer of ribonucleic acid (RNA) or deoxyribonucleic acid (DNA), or RNA-like or DNA-like oligomers. U.S. Pat. Nos. 5,898,031 and 6,107,094, each of which is herein incorporated by reference, describe certain oligonucleotides having RNA-like properties. When hybridized with RNA, these oligonucleotides serve as substrates for a dsRNase enzyme with resultant cleavage of the RNA by the enzyme (Crooke, 2000; Crooke, 1999). An example of an RNA-like includes an RNA molecule having 2′-hydroxyl or 2′-modified sugars. In some embodiments, the single-stranded oligonucleotides may be “DNA-like”, in that the oligonucleotide has well characterized structural features, for example a plurality of unmodified 2′ Hs or a stabilized backbone such as e.g., phosphorothioate, that is structurally suited for interaction with a target oligonucleotide and recruitment and (activation) of RNase H. The oligomer may be single-stranded, or may be a self-hybridizing, or constructs comprising an antisense orientation oligonucleotide. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

The antisense compounds in accordance with this invention preferably comprise from about 8 to about 30 nucleobases, more preferably from about 12 to about 28 and most preferably from about 15 to about 26 nucleobases. Particularly preferred antisense compounds are antisense oligonucleotides. A discussion of antisense oligonucleotides and some desirable modifications can be found in De Mesmaeker et al., Acc. Chem. Res., 1995, 28, 366.

An oligonucleotide is a polymer of a repeating unit generically known as a nucleotide. An unmodified (naturally occurring) nucleotide has three components: (1) a nitrogen-containing heterocyclic base linked by one of its nitrogen atoms to (2) a 5-pentofuranosyl sugar and (3) a phosphate esterified to one of the 5′ or 3′ carbon atoms of the sugar. When incorporated into an oligonucleotide chain, the phosphate of a first nucleotide is also esterified to an adjacent sugar of a second, adjacent nucleotide via a 3′-5′ phosphate linkage.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be further joined to form a circular structure, however, within the context of the invention, open linear structures are generally preferred.

Within the oligonucleotide structure, the phosphate groups are commonly referred to as forming the intersugar “backbone” of the oligonucleotide. The normal linkage or backbone of RNA and DNA is a 3′ to 5′ phosphodiester linkage. The backbone of an oligonucleotide (or other antisense compound) positions a series of bases in a specific order; the written representation of this ordered series of bases, usually written in 5′ to 3′ order unless otherwise indicated, is known as a nucleotide or nucleobase sequence.

Oligonucleotides may comprise nucleotide sequences sufficient in identity and number to effect specific hybridization with a particular nucleic acid. Such oligonucleotides which specifically hybridize to a portion of the sense strand of a gene are commonly described as “antisense.” In the context of the invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other.

“Specifically hybridizable” and “complementary” are thus terms which are used to indicate a sufficient degree of complementarity or precise pairing such that stable and specific binding occurs between the oligonucleotide and the DNA or RNA target. An oligonucleotide is specifically hybridizable to its target sequence due to the formation of base pairs between specific partner nucleobases in the interior of a nucleic acid duplex. Among the naturally occurring nucleobases, guanine (G) binds to cytosine (C), and adenine (A) binds to thymine (T) or uracil (U). In addition to the equivalency of U (RNA) and T (DNA) as partners for A, other naturally occurring nucleobase equivalents are known, including 5-methylcytosine and 5-hydroxymethylcytosine (HMC) (C equivalents), and 5-hydroxymethyluracil (U equivalent). Furthermore, synthetic nucleobases which retain partner specificity are known in the art and include, for example, 7-deaza-Guanine, which retains partner specificity for C. Thus, an oligonucleotide's capacity to specifically hybridize with its target sequence will not be altered by a chemical modification to a nucleobase in the nucleotide sequence of the oligonucleotide which does not impact its specificity for a partner nucleobase in the target nucleic acid.

It is understood in the art that the nucleobase sequence of an oligonucleotide or other antisense compound need not be 100% complementary to its target nucleic acid sequence to be specifically hybridizable. An antisense compound is specifically hybridizable to its target nucleic acid when there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under assay conditions.

Antisense oligonucleotides are commonly used as research reagents, diagnostic aids, and therapeutic agents. For example, antisense oligonucleotides, which are able to inhibit gene expression with exquisite specificity, are often used by those of ordinary skill to elucidate the function of particular genes, for example to distinguish between the functions of various members of a biological pathway. This specific inhibitory effect has, therefore, been harnessed by those skilled in the art for research uses. The specificity and sensitivity of oligonucleotides is also harnessed by those of skill in the art for therapeutic uses. Specific examples of preferred antisense compounds useful in this invention include oligonucleotides containing modified backbones or non-natural intersugar linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their intersugar backbone can also be considered to be oligonucleosides.

Specific oligonucleotide chemical modifications are described in the following subsections. It is not necessary for all positions in a given compound to be uniformly modified, and in fact more than one of the following modifications may be incorporated in a single antisense compound or even in a single residue thereof, for example, at a single nucleoside within an oligonucleotide.

Modified Linkages: Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Representative U.S. patents that teach the preparation of the above phosphorus atom containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248, certain of which are commonly owned with this application, and each of which is herein incorporated by reference.

Preferred modified oligonucleotide backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, certain of which are commonly owned with this application, and each of which is herein incorporated by reference.

In other preferred oligonucleotide mimetics, both the sugar and the intersugar linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al. (Science, 1991, 254, 1497).

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified Nucleobases: The compounds of the invention may additionally or alternatively comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2 degrees C. (Id., pages 276-278) and are presently preferred base substitutions, even more particularly when combined with 2′-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, certain of which are commonly owned, and each of which is herein incorporated by reference, and commonly owned U.S. patent application 08/762,488, filed on Dec. 10, 1996, also herein incorporated by reference.

Sugar Modifications: The antisense compounds of the invention may additionally or alternatively comprise one or more substituted sugar moieties. Preferred oligonucleotides comprise one of the following at the 2′ position: OH; F; O-, S-, or N-alkyl, O-, S-, or N-alkenyl, or O, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy [2′-O—CH₂CH₂OCH₃, also known as 2′-O-(2-methoxyethyl) or 2′-MOE] (Martin et al., Helv. Chim. Acta, 1995, 78, 486), i.e., an alkoxyalkoxy group. A further preferred modification includes 2′-dimethylaminooxyethoxy, i.e., a O(CH₂)₂ON(CH₃)₂ group, also known as 2′-DMAOE, as described in co-owned U.S. patent application Ser. No. 09/016,520, filed on Jan. 30, 1998, the contents of which are herein incorporated by reference.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative U.S. patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053l 5,639,873; 5,646,265; 5,658,873; 5,670,633; and 5,700,920, certain of which are commonly owned, and each of which is herein incorporated by reference, and commonly owned U.S. patent application Ser. No. 08/468,037, filed on Jun. 5, 1995, also herein incorporated by reference.

Other Modifications: Additional modifications may also be made at other positions on the oligonucleotide, particularly the 3′ position of the sugar on the 3′ terminal nucleotide and the 5′ position of 5′ terminal nucleotide. For example, one additional modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned, and each of which is herein incorporated by reference.

Chimeric Oligonucleotides: The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate oligodeoxynucleotides hybridizing to the same target region. Cleavage of the RNA target can be routinely detected by gel electrophoresis and, if necessary, associated nucleic acid hybridization techniques known in the art. RNase H-mediated target cleavage is distinct from the use of ribozymes to cleave nucleic acids, and ribozymes are not comprehended by the present invention.

By way of example, such “chimeras” may be “gapmers,” i.e., oligonucleotides in which a central portion (the “gap”) of the oligonucleotide serves as a substrate for, e.g., RNase H, and the 5′ and 3′ portions (the “wings”) are modified in such a fashion so as to have greater affinity for, or stability when duplexed with, the target RNA molecule but are unable to support nuclease activity (e.g., 2′-fluoro- or 2′-methoxyethoxy- substituted). Other chimeras include “hemimers,” that is, oligonucleotides in which the 5′ portion of the oligonucleotide serves as a substrate for, e.g., RNase H, whereas the 3′ portion is modified in such a fashion so as to have greater affinity for, or stability when duplexed with, the target RNA molecule but is unable to support nuclease activity (e.g., 2′-fluoro- or 2′-methoxyethoxy- substituted), or vice-versa.

A number of chemical modifications to oligonucleotides that confer greater oligonucleotide:RNA duplex stability have been described by Freier et al. (Nucl. Acids Res., 1997, 25, 4429). Such modifications are preferred for the RNase H-refractory portions of chimeric oligonucleotides and may generally be used to enhance the affinity of an antisense compound for a target RNA.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned, and each of which is herein incorporated by reference, and commonly owned and allowed U.S. patent application Ser. No. 08/465,880, filed on Jun. 6, 1995, also herein incorporated by reference.

Examples of specific oligonucleotides and the target genes to which they inhibit which may be employed in formulations of the present invention include:

| | | | |
|---|---|---|---|
| ISIS-2302 | GCCCA AGCTG GCATC CGTCA | (SEQ ID NO:1) | ICAM-1 |
| ISIS-15839 | GCCCA AGCTG GCATC CGTCA | (SEQ ID NO:1) | ICAM-1 |
| ISIS-2922 | GCGTT TGCTC TTCTT CTTGC G | (SEQ ID NO:2) | HCMV |
| ISIS-13312 | GCGTT TGCTC TTCTT CTTGC G | (SEQ ID NO:2) | HCMV |
| ISIS-3521 | GTTCT CGCTG GTGAG TTTCA | (SEQ ID NO:3) | PKC |
| ISIS-9605 | GTTCT CGCTG GTGAG TTTCA | (SEQ ID NO:3) | PKC |
| ISIS-9606 | GTTCT CGCTG GTGAG TTTCA | (SEQ ID NO:3) | PKC |
| ISIS-14859 | AACTT GTGCT TGCTC | (SEQ ID NO:4) | PKC |
| ISIS-2503 | TCCGT CATCG CTCCT CAGGG | (SEQ ID NO:5) | Ha-ras |
| ISIS-5132 | TCCCG CCTGT GACAT GCATT | (SEQ ID NO:6) | c-raf |
| ISIS-5320 | TTGGG GTT | | gp120 |
| ISIS-14803 | GTGCT CATGG TGCAC GGTCT | (SEQ ID NO:7) | HCV |
| ISIS-17044 | CCGCA GCCAT GCGCT CTTGG | (SEQ ID NO:8) | VLA-4 |
| ISIS-28089 | GTGTG CCAGA CACCC TATCT | (SEQ ID NO:9) | TNF |
| ISIS-104838 | GCTGA TTAGA GAGAG GTCCC | (SEQ ID NO:10) | TNF |
| ISIS-2105 | TTGCT TCCAT CTTCC TCGTC | (SEQ ID NO:11) | HPV |
| ISIS-3082 | TGCAT CCCCC AGGCC ACCAT | (SEQ ID NO:12) | ICAM-1 |

wherein (i) each oligo backbone linkage is a phosphorothioate linkage (except ISIS-9605) and (ii) each sugar is 2′-deoxy unless represented in bold font in which case it incorporates a 2′-O-methoxyethyl group and iii) underlined cytosine nucleosides incorporate a 5-methyl substituent on their nucleobase. ISIS-9605 incorporates natural phosphodiester bonds at the first five and last five linkages with the remainder being phosphorothioate linkages.

Other Bioactive oligomers: The term “Other Bioactive oligomer” encompasses, inter alia, aptamers and molecular decoys. As used herein, the term is meant to refer to any oligonucleotide (including an RNA or PNA) that (1) provides a prophylactic, palliative or therapeutic effect to an animal in need thereof and (2) acts by a non-antisense mechanism, i.e., by some means other than by hybridizing to a nucleic acid.

Aptamers are single-stranded oligonucleotides that bind specific ligands via a mechanism other than Watson-Crick base pairing. Aptamers are typically targeted to, e.g., a protein and are not designed to bind to a nucleic acid (Ellington et al., Nature, 1990, 346, 818).

Molecular decoys are short double-stranded nucleic acids (including single-stranded nucleic acids designed to “fold back” on themselves) that mimic a site on a nucleic acid to which a factor, such as a protein, binds. Such decoys are expected to competitively inhibit the factor; that is, because the factor molecules are bound to an excess of the decoy, the concentration of factor bound to the cellular site corresponding to the decoy decreases, with resulting therapeutic, palliative or prophylactic effects. Methods of identifying and constructing decoy molecules are described in, e.g., U.S. Pat. No. 5,716,780 to Edwards et al.

Another type of bioactive oligomer is an RNA-DNA hybrid molecule that can direct gene conversion of an endogenous nucleic acid (Cole-Strauss et al., Science, 1996, 273, 1386). Any of the preceding bioactive oligomers may be formulated in the liposomes of the invention and used for prophylactic or therapeutic purposes.

Some embodiments of the invention, a single oligonucleotide having both the antisense portion as a first region in the oligonucleotide and the sense portion as a second region in the oligonucleotide is selected. The first and second regions are linked together by either a nucleotide linker (a string of one or more nucleotides that are linked together in a sequence) or by a non-nucleotide linker region or by a combination of both a nucleotide and non-nucleotide structure. In each of these structures, the oligonucleotide, when folded back on itself, would be complementary at least between the first region, the antisense portion, and the second region, the sense portion. Thus the oligonucleotide would have a palindrome within it structure wherein the first region, the antisense portion in the 5′ to 3′ direction, is complementary to the second region, the sense portion in the 3′ to 5′ direction.

In further embodiments, the invention includes an oligonucleotide/protein composition. This composition has both an oligonucleotide component and a protein component. The oligonucleotide component comprises at least one oligonucleotide, either the antisense or the sense oligonucleotide but preferable the antisense oligonucleotide (the oligonucleotide that is antisense to the target nucleic acid). The protein component of the composition comprises at least one protein that forms a portion of the RNA-induced silencing complex, i.e., the RISC complex. The oligonucleotide component can also comprise both the antisense and the sense strand oligonucleotides.

RISC is a ribonucleoprotein complex that contains an oligonucleotide component and proteins of the Argonaute family of proteins. While we do not wish to be bound by theory, the Argonaute proteins are a class of proteins, some of which have been shown to contain a PAZ and Piwi domain and that have been implicated in processes previously linked to posttranscriptional silencing. The Argonaute family of proteins includes, but depending on species, are not necessary limited to e1F2C1 and e1F2C2. e1F2C2 is also known as human GERp95. While we do not wish to be bound by theory, at least the antisense oligonucleotide strand is bound to the protein component to form the RISC complex. Additional, the complex might also include the sense strand oligonucleotide.

Also while not bound by theory, it is further believe that the RISC complex interacts with the ribosomes or polyribosome complex. Therefore, in an embodiment of the invention, the invention also includes association of the oligonucleotide component of the invention with the RISC protein component and further associated with and interact with the translation machinery of a cell. Such interaction with the translation machinery of the cell would include interaction with structural and enzymatic proteins of the translation machinery including but not limited to the polyribosome and ribosomal subunits. These subunits include but not necessary limited to eIF2Cs and eIF4s.

The oligomeric compounds of the invention may be used in the form of single-stranded, double-stranded, circular or hairpin oligomeric compounds and may contain structural elements such as internal or terminal bulges or loops. Once introduced to a system, the oligomeric compounds of the invention may elicit the action of one or more enzymes or proteins to effect modification of the target nucleic acid.

One non-limiting example of such a protein is the RISC complex. Use of the RISC complex to effect cleavage of RNA targets thereby greatly enhances the efficiency of oligonucleotide-mediated inhibition of gene expression. Similar roles have been postulated for other ribonucleases such as those in the RNase III and ribonuclease L family of enzymes.

Preferred forms of oligomeric compound of the invention include a single-stranded antisense oligonucleotide that binds in a RISC complex, a double stranded antisense/sense pair of oligonucleotide or a single strand oligonucleotide that includes both an antisense portion and a sense portion. Each of these compounds or compositions is used to induce potent and specific modulation of gene function. Such specific modulation of gene function has been shown in many species by the introduction of double-stranded structures, such as double-stranded RNA (dsRNA) molecules and has been shown to induce potent and specific antisense-mediated reduction of the function of a gene or its associated gene products. This phenomenon occurs in both plants and animals and is believed to have an evolutionary connection to viral defense and transposon silencing.

A further preferred modification includes 2′-dimethylamino oxyethoxy, i.e., a O(CH₂)₂ON(CH₃)₂ group, also known as 2′-DMAOE, as described in co-owned U.S. patent application Ser. No. 09/016,520, filed on Jan. 30, 1998, the contents of which are herein incorporated by reference. Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the sugar group, particularly the 3′ position of the sugar on the 3′ terminal nucleotide or in 2′-5′ linked oligonucleotides and the 5′ position of 5′ terminal nucleotide. The nucleosides of the oligonucleotides may also have sugar mimetics such as cyclobutyl moieties in place of the pentofuranosyl sugar.

Unsubstituted and substituted phosphodiester oligonucleotides are alternately synthesized on an automated DNA synthesizer (Applied Biosystems model 380B) using standard phosphoramidite chemistry with oxidation by iodine.

Phosphorothioates are synthesized as per the phosphodiester oligonucleotides except the standard oxidation bottle was replaced by 0.2 M solution of 3H-1,2-benzodithiole-3-one 1,1-dioxide in acetonitrile for the stepwise thiation of the phosphite linkages. The thiation wait step was increased to 68 sec and was followed by the capping step. After cleavage from the CPG column and deblocking in concentrated ammonium hydroxide at 55° C. (18 hr), the oligonucleotides were purified by precipitating twice with 2.5 volumes of ethanol from a 0.5 M NaCl solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No. 5,256,775 or U.S. Pat. No. 5,366,878, hereby incorporated by reference.

Alkylphosphonothioate oligonucleotides are prepared as described in published PCT applications PCT/US94/00902 and PCT/US93/06976 (published as WO 94/17093 and WO 94/02499, respectively).

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Boranophosphate oligonucleotides are prepared as described in U.S. Pat. Nos. 5,130,302 and 5,177,198, both herein incorporated by reference.

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and PO or PS linkages are prepared as described in U.S. Pat. Nos. 5,378,825; 5,386,023; 5,489,677; 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5. They may also be prepared in accordance with U.S. Pat. Nos. 5,539,082; 5,700,922, and 5,719,262, herein incorporated by reference.

Synthesis of Oligonucleotides: The oligonucleotides used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides such as the phosphorothioates and alkylated derivatives.

Teachings regarding the synthesis of particular modified oligonucleotides may be found in the following U.S. patents or pending patent applications, each of which is commonly assigned with this application: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having -lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. Nos. 5,223,168, issued Jun. 29, 1993, and 5,608,046, both drawn to conjugated 4′-desmethyl nucleoside analogs; U.S. Pat. Nos. 5,602,240, and 5,610,289, drawn to backbone modified oligonucleotide analogs; and U.S. patent application Ser. No. 08/383,666, filed Feb. 3, 1995, and U.S. Pat. No. 5,459,255, drawn to, inter alia, methods of synthesizing 2′-fluoro-oligonucleotides.

B. Non-Oligonucleotidic Anticancer Agents (a.k.a. chemotherapeutic agents) that may be incorporated within the liposomes and pharmaceutical compositions of the invention include, but are not limited to, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, nitrogen mustards, methylcyclohexylnitrosurea, melphalan, cyclophosphamide, 6-mercaptopurine, 6-thioguanine, cytarabine (CA), 5-azacytidine, hydroxyurea, 5-fluorouracil (5-FU), 4-hydroxyperoxycyclophosphoramide, 5-fluorodeoxyuridine (5-FUdR), methotrexate (MTX), colchicine, deoxycoformycin, vincristine, vinblastine, etoposide, trimetrexate, teniposide, cisplatin and diethylstilbestrol (DES). See, generally, The Merck Manual of Diagnosis and Therapy, 15th Ed., pp. 1206-1228, Berkow et al., eds., Rahway, N.J., 1987).

Other non-oligonucleotidic anticancer and/or antimetastatic agents that may be incorporated within the liposomes and pharmaceutical compositions of the invention include, e.g., inhibitors of stress activated protein kinases (SAPKs) or agents that inhibit factors that positively regulate and/or activate SAPKs (such as those described in U.S. Pat. Nos. 5,593,992, 5,593,991, 5,670,527, 5,658,903, 5,656,644, 5,559,137 and 5,525,625, as well as those described in published PCT applications WO 97/25048, WO 97/25047, WO 97/25046, WO 97/25045, WO 96/40143 and WO 96/21654), inhibitors of metalloproteinases (such as are described in U.S. Pat. No. 5,602,156 to Kohn et al.) and the like.

C. Non-Oligonucleotidic Anti-Inflammatory Agents may also be incorporated within the liposomal compositions of the invention. Such agents include, but are not limited to, salicylates; nonsteroidal anti-inflammatory drugs (NSAIDs), including indomethacin, ibuprofen, fenopofen, ketoprofen, naproxen, piroxicam, phenylbutazone, oxyphenbutazone, sulindac and meclofenamate; gold compounds, such as auranofin; D-penicillamine; cyclophosphamide; methotrexate; azathioprine; colchicine; hydroxychloroquine; corticotropin; steroids and corticosteroids such as, for example, hydrocortisone, deoxyhydrocortisone, fluorocortisone, prednisolone, methylprednisolone, prednisone, triamcinolone, dexamethasone, betamethasone and paramethasone. See, generally, The Merck Manual of Diagnosis and Therapy, 15th Ed., pp. 1239-1267 and 2497-2506, Berkow et al., eds., Rahay, N.J., 1987.

D. Combinations of Bioactive Agents are developed with the following considerations in mind. In some cases it may be more effective to treat a patient with a liposomal composition of the invention in conjunction with other traditional therapeutic modalities in order to increase the efficacy of a treatment regimen. In the context of the invention, the term “treatment regimen” is meant to encompass therapeutic, palliative and prophylactic modalities. For example, a patient may be treated with conventional chemotherapeutic agents, particularly those used for tumor and cancer treatment, or anti-inflammatory agents (examples of such agents are given supra), in combination with a liposomal composition of the invention comprising a bioactive agent. As another example, a patient may be treated with two or more bioactive agents, both of which are incorporated within a liposomal composition of the invention.

When combinations of bioactive agents, one or more of which are encapsulated in the liposomes of the invention, are used, the agents may be concomitantly administered as separate pharmaceutical compositions, co-administered as a combined pharmaceutical composition, administered sequentially or in combination with one or more other anticancer agents or physical treatments (e.g., radiotherapy for anticancer therapies).

E. Bioequivalents: The bioactive agents of the invention encompass any pharmaceutically acceptable compounds which, upon administration to an animal including a human, is capable of providing (directly or indirectly) the biologically active metabolite or residue thereof. Accordingly, for example, the disclosure is also drawn to “prodrugs” and “pharaceutically acceptable salts” of the antisense compounds of the invention and other bioequivalents.

Prodrugs: Bioactive agents encapsulated by the liposomes of the invention may additionally or alternatively be prepared to be delivered in a “prodrug” form. The term “prodrug” indicates a therapeutic compound that is prepared in an inactive form that is converted to an active form (i.e., drug) within the body or cells thereof by the action of endogenous enzymes or other chemicals and/or conditions. In particular, prodrug versions of the antisense compounds of the invention are prepared as SATE [(S-acetyl-2-thioethyl) phosphate] derivatives according to the methods disclosed in WO 93/24510 to Gosselin et al.

Pharmaceutically Acceptable Salts: The term “pharmaceutically acceptable salts” refers to physiologically and pharmaceutically acceptable salts of the oligonucleotide and nucleic acid compounds employed in the compositions of the present invention (i.e., salts that retain the desired biological activity of the parent compound and do not impart undesired toxicological effects thereto).

Pharmaceutically acceptable base addition salts are formed with metals or amines, such as alkali and alkaline earth metals or organic amines. Examples of metals used as cations are sodium, potassium, magnesium, calcium, ammonium, polyamines such as spermine and spermidine, and the like. Examples of suitable amines are chloroprocaine, choline, N,N′-dibenzylethylenediamine, diethanolamine, dicyclohexylamine, ethylenediamine, N-methylglucamine, and procaine (see, for example, Berge et al., “Pharmaceutical Salts,” J. of Pharma Sci., 1977, 66: 1). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

Oligonucleotide Deletion Derivatives: During the process of oligonucleotide synthesis, nucleoside monomers are attached to the chain one at a time in a repeated series of chemical reactions such as nucleoside monomer coupling, oxidation, capping and detritylation. The stepwise yield for each nucleoside addition is above 99%. That means that less than 1% of the sequence chain failed to the nucleoside monomer addition in each step as the total results of the incomplete coupling followed by the incomplete capping, detritylation and oxidation (Smith, Anal. Chem., 1988, 60, 381A). All the shorter oligonucleotides, ranging from (n−1), (n−2), etc., to 1-mers (nucleotides), are present as impurities in the n-mer olignucleotide product. Among the impurities, (n−2)-mer and shorter oligonucleotide impurities are present in very small amounts and can be easily removed by chromatographic purification (Warren et al., Chapter 9 In: Methods in Molecular Biology, Vol. 26: Protocols for Oligonucleotide Conjugates, Agrawal, S., Ed., 1994, Humana Press Inc., Totowa, N.J., pages 233-264). However, due to the lack of chromatographic selectivity and product yield, some (n−1)-mer impurities are still present in the full-length (i.e., n-mer) oligonucleotide product after the purification process. The (n−1) portion consists of the mixture of all possible single base deletion sequences relative to the n-mer parent oligonucleotide. Such (n−1) impurities can be classified as terminal deletion or internal deletion sequences, depending upon the position of the missing base (i.e., either at the 5′ or 3′ terminus or internally). When an oligonucleotide containing single base deletion sequence impurities is used as a drug (Crooke, Hematologic Pathology, 1995, 9, 59), the terminal deletion sequence impurities will bind to the same target mRNA as the full length sequence but with a slightly lower affinity. Thus, to some extent, such impurities can be considered as part of the active drug component, and are thus considered to be bioequivalents for purposes of the present invention.

IV. Pharmaceutical Compositions

The present invention provides for therapeutic and pharmaceutical compositions comprising liposomes in which bioactive agents are incorporated. Compositions for the administration of the liposomal compositions of the invention may include sterile aqueous solutions which may also contain buffers, diluents and other suitable additives.

The pharmaceutical compositions of the invention, which may conveniently be presented in unit dosage form, may be prepared according to conventional techniques well known in the pharmaceutical industry. Such techniques include the step of bringing into association the active ingredient(s) with the pharmaceutically acceptable carrier(s). In general the pharmaceutical compositions are prepared by uniformly and intimately bringing into association the active ingredient(s) with liquid excipients or finely divided solid excipients or both, and then, if necessary, shaping the product.

Pharmaceutical and therapeutic compositions comprising one or more of the antisense compounds of the invention may further include sterile aqueous solutions which may also contain buffers, diluents and other suitable additives. Pharmaceutically acceptable organic or inorganic carrier substances suitable for non-parenteral administration which do not deleteriously react with the antisense compounds can be used. The pharmaceutical compositions can be sterilized and, if desired, mixed with auxiliary agents, e.g., lubricants, preservatives, stabilizers, wetting agents, emulsifiers, salts for influencing osmotic pressure, buffers, colorings flavorings and/or aromatic substances and the like which do not deleteriously react with the oligonucleotide(s) of the pharmaceutical composition. Pharmaceutical compositions in the form of aqueous suspensions may contain substances which increase the viscosity of the suspension including, for example, sodium carboxymethylcellulose, sorbitol and/or dextran. Optionally, such compositions may also contain one or more stabilizers, carrier compounds or pharmaceutically acceptable carriers.

Carrier Compounds: As used herein, “carrier compound” refers to a compound, which is inert (i.e., does not possess biological activity per se) but is recognized by in vivo processes that reduce the bioavailability of a bioactive agent by, for example, degrading the bioactive agent or promoting its removal from circulation. The coadministration of a bioactive agent and a carrier compound, typically with an excess of the latter substance, can result in a substantial reduction of the amount of bioactive agent recovered in the liver, kidney or other extracirculatory reservoirs, presumably due to competition between the carrier compound and the bioactive agent for a common receptor. For example, the recovery of a partially phosphorothioated oligonucleotide in hepatic tissue is reduced when it is coadministered with polyinosinic acid, dextran sulfate, polycytidic acid or 4-acetamido-4′-isothiocyano-stilbene-2,2′-disulfonic acid (Miyao et al., Antisense Res. Dev., 1995, 5, 115; Takakura et al., Antisense & Nucl. Acid Drug Dev., 1996, 6, 177).

Pharmaceutically Acceptable Carriers: In contrast to a carrier compound, a “pharmaceutically acceptable carrier” (excipient) is a pharmaceutically acceptable solvent, suspending agent or any other pharmacologically inert vehicle for delivering one or more bioactive agents to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Suitable pharmaceutically acceptable carriers include, but are not limited to, water, salt solutions, alcohol, polyethylene glycols, gelatin, lactose, amylose, magnesium stearate, talc, silicic acid, hydroxymethylcellulose, polyvinylpyrrolidone viscous paraffin and the like.

Miscellaneous Additional Components: The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

V. Methods of Administration

The administration of therapeutic or pharmaceutical compositions comprising the liposomes of the invention is believed to be within the skill of those in the art. In general, a patient in need of therapy or prophylaxis is administered a composition comprising a liposomally formulated bioactive agents in accordance with the invention, commonly in a pharmaceutically acceptable carrier, in doses ranging from 0.01 ug to 100 g per kg of body weight depending on the age of the patient and the severity of the disorder or disease state being treated. Dosing is dependent on severity and responsiveness of the disease state to be treated, with the course of treatment lasting from several days to several months, or until a cure is effected or a diminution or prevention of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual antisense compounds, and can generally be estimated based on EC₅₀s found to be effective in in vitro and in vivo animal models.

Treatment Regimens: In the context of the invention, the term “treatment regimen” is meant to encompass therapeutic, palliative and prophylactic modalities of administration of one or more liposomal compositions of the invention. A particular treatment regimen may last for a period of time which will vary depending upon the nature of the particular disease or disorder, its severity and the overall condition of the patient, and may extend from once daily to once every 20 years. Following treatment, the patient is monitored for changes in his/her condition and for alleviation of the symptoms of the disorder or disease state. The dosage of the liposomal composition may either be increased in the event the patient does not respond significantly to current dosage levels, or the dose may be decreased if an alleviation of the symptoms of the disorder or disease state is observed, or if the disorder or disease state has been ablated.

An optimal dosing schedule is used to deliver a therapeutically effective amount of the bioactive agent encapsulated within the liposomes of the invention being administered via a particular mode of administration. The term “therapeutically effective amount,” for the purposes of the invention, refers to the amount of oligonucleotide-containing pharmaceutical composition which is effective to achieve an intended purpose without undesirable side effects (such as toxicity, irritation or allergic response). Although individual needs may vary, determination of optimal ranges for effective amounts of pharmaceutical compositions is within the skill of the art. Human doses can be extrapolated from animal studies (Katocs et al., Chapter 27 In: Remington's Pharmaceutical Sciences, 18th Ed., Gennaro, ed., Mack Publishing Co., Easton, Pa., 1990). Generally, the dosage required to provide an effective amount of a pharmaceutical composition, which can be adjusted by one skilled in the art, will vary depending on the age, health, physical condition, weight, type and extent of the disease or disorder of the recipient, frequency of treatment, the nature of concurrent therapy (if any) and the nature and scope of the desired effect(s) (Nies et al., Chapter 3 In: Goodman & Gilman's The Pharmacological Basis of Therapeutics, 9th Ed., Hardman et al., eds., McGraw-Hill, New York, N.Y., 1996).

Following successful treatment, it may be desirable to have the patient undergo maintenance therapy to prevent the recurrence of the disease state, wherein the bioactive agent is administered in maintenance doses, ranging from 0.01 ug to 100 g per kg of body weight, once or more daily, to once every 20 years. For example, in the case of in individual known or suspected of being prone to an autoimmune or inflammatory condition, prophylactic effects may be achieved by administration of preventative doses, ranging from 0.01 ug to 100 g per kg of body weight, once or more daily, to once every 20 years. In like fashion, an individual may be made less susceptible to an inflammatory condition that is expected to occur as a result of some medical treatment, e.g., graft versus host disease resulting from the transplantation of cells, tissue or an organ into the individual.

Prophylactic modalities for high risk individuals are also encompassed by the invention. As used herein, the term “high risk individual” is meant to refer to an individual for whom it has been determined, via, e.g., individual or family history or genetic testing, that there is a significantly higher than normal probability of being susceptible to the onset or recurrence of a disease or disorder. As part of a treatment regimen for a high risk individual, the individual can be prophylactically treated to prevent the onset or recurrence of the disease or disorder. The term “prophylactically effective amount” is meant to refer to an amount of a pharmaceutical composition which produces an effect observed as the prevention of the onset or recurrence of a disease or disorder. Prophylactically effective amounts of a pharmaceutical composition are typically determined by the effect they have compared to the effect observed when a second pharmaceutical composition lacking the active agent is administered to a similarly situated individual.

The therapeutic and pharmaceutical compositions of the present invention may be administered in a number of ways depending upon whether local or systemic treatment is desired and upon the area to be treated. Typically, parenteral administration is employed. The term “parenteral delivery” refers to the administration of one or more antisense compounds of the invention to an animal in a manner other than through the digestive canal. Parenteral administration includes intravenous (i.v.) drip, subcutaneous, intraperitoneal (i.p.) or intramuscular injection, or intrathecal or intraventricular administration. Compositions for parenteral, intrathecal or intraventricular administration may include sterile aqueous solutions which may also contain buffers, diluents and other suitable additives. Means of preparing and administering parenteral pharmaceutical compositions are known in the art (see, e.g., Avis, Chapter 84 In: Remington's Pharmaceutical Sciences, 18th Ed., Gennaro, ed., Mack Publishing Co., Easton, Pa., 1990, pages 1545-1569). Parenteral means of delivery include, but are not limited to, the following illustrative examples.

Intravenous administration of pharmaceutical formulations comprising bioactive agents, including liposomes encapsulating bioactive agents is well known in the art. Bioactive agents encapsulated in liposomes and delivered i.v. include an anticancer drug, doxorubicin, and an antifungal drug, amphotericin B (see, e.g., Riaz et al., Chapter 16 in Pharmaceutical Dosage Forms: disperse Systems, Vol. 2, Lieberman et al., eds., Marcel Dekker, Inc., New York, N.Y., 1989, pages 567-603). With particular regards to antisense compounds, i.v. administration of such compounds (not encapsulated in liposomes) to various non-human mammals has been described by Iversen (Chapter 26 In: Antisense Research and Applications, Crooke et al., eds., CRC Press, Boca Raton, Fla., 1993, pages 461-469), and systemic delivery of oligonucleotides to non-human mammals via intraperitoneal means has also been described (Dean et al., Proc. Natl. Acad. Sci. USA, 1994, 91, 11766).

Intraluminal drug administration, for the direct delivery of drug to an isolated portion of a tubular organ or tissue (e.g., such as an artery, vein, ureter or urethra), may be desired for the treatment of patients with diseases or conditions afflicting the lumen of such organs or tissues. To effect this mode of administration, a catheter or cannula is surgically introduced by appropriate means. For example, for treatment of the left common carotid artery, a cannula is inserted thereinto via the external carotid artery. After isolation of a portion of the tubular organ or tissue for which treatment is sought, a composition comprising the antisense compounds of the invention is infused through the cannula or catheter into the isolated segment. After incubation for from about 1 to about 120 minutes, during which the bioactive agent is taken up by cells of the interior lumen of the vessel, the infusion cannula or catheter is removed and flow within the tubular organ or tissue is restored by removal of the ligatures which effected the isolation of a segment thereof (Morishita et al., Proc. Natl. Acad. Sci. U.S.A., 1993, 90, 8474). Bioactive agents may also be combined with a biocompatible matrix, such as a hydrogel material, and applied directly to vascular tissue in vivo (Rosenberg et al., U.S. Pat. No. 5,593,974, issued Jan. 14, 1997).

Intraventricular drug administration, for the direct delivery of drug to the brain of a patient, may be desired for the treatment of patients with diseases or conditions afflicting the brain. To effect this mode of administration, a silicon catheter is surgically introduced into a ventricle of the brain of a human patient, and is connected to a subcutaneous infusion pump (Medtronic Inc., Minneapolis, Minn.) that has been surgically implanted in the abdominal region (Zimm et al., Cancer Research, 1984, 44, 1698; Shaw, Cancer, 1993, 72(11 Suppl., 3416). The pump is used to inject the bioactive agent and allows precise dosage adjustments and variation in dosage schedules with the aid of an external programming device. The reservoir capacity of the pump is 18-20 mL and infusion rates may range from 0.1 mL/h to 1 mL/h. Depending on the frequency of administration, ranging from daily to monthly, and the dose of drug to be administered, ranging from 0.01 ug to 100 g per kg of body weight, the pump reservoir may be refilled at 3-10 week intervals. Refilling of the pump is accomplished by percutaneous puncture of the pump's self-sealing septum.

Intrathecal drug administration, for the introduction of a drug into the spinal column of a patient may be desired for the treatment of patients with diseases of the central nervous system (CNS). To effect this route of administration, a silicon catheter is surgically implanted into the L3-4 lumbar spinal interspace of a human patient, and is connected to a subcutaneous infusion pump which has been surgically implanted in the upper abdominal region (Luer and Hatton, The Annals of Pharmacotherapy, 1993, 27, 912, 1993; Ettinger et al. Cancer, 1978, 41, 1270; Yaida et al., Regul. Pept., 1985, 59, 193). The pump is used to inject the bioactive agent and allows precise dosage adjustments and variations in dose schedules with the aid of an external programming device. The reservoir capacity of the pump is 18-20 mL, and infusion rates may vary from 0.1 mL/h to 1 mL/h. Depending on the frequency of drug administration, ranging from daily to monthly, and dosage of drug to be administered, ranging from 0.01 ug to 100 g per kg of body weight, the pump reservoir may be refilled at 3-10 week intervals. Refilling of the pump is accomplished by a single percutaneous puncture to the self-sealing septum of the pump. The distribution, stability and pharmacokinetics of a bioactive agent (such as an oligonucleotide) within the CNS are followed according to known methods (Whitesell et al., Proc. Natl. Acad. Sci. USA, 1993, 90, 4665).

To effect delivery of bioactive agents to areas other than the brain or spinal column via this method, the silicon catheter is configured to connect the subcutaneous infusion pump to, e.g., the hepatic artery, for delivery to the liver (Kemeny et al., Cancer, 1993, 71, 1964). Infusion pumps may also be used to effect systemic delivery of bioactive agents (Ewel et al., Cancer Res., 1992, 52, 3005; Rubenstein et al., J. Surg. Oncol., 1996, 62, 194).

Vaginal Delivery provides local treatment and avoids first pass metabolism, degradation by digestive enzymes, and potential systemic side-effects. This mode of administration may be preferred for bioactive agents targeted to pathogenic organisms for which the vagina is the usual habitat, e.g., Trichomonas vaginalis. Vaginal suppositories (Block, Chapter 87 In: Remington's Pharmaceutical Sciences, 18th Ed., Gennaro, ed., Mack Publishing Co., Easton, Pa., 1990, pages 1609-1614) or topical ointments can be used to effect this mode of delivery.

Intravesical Delivery provides local treatment and avoids first pass metabolism, degradation by digestive enzymes, and potential systemic side-effects. However, the method requires urethral catheterization of the patient and a skilled staff. Nevertheless, this mode of administration may be preferred for bioactive agents targeted to pathogenic organisms, such as T. vaginalis, which may invade the urogenital tract.

Intravitreal injection, for the direct delivery of drug to the vitreous humor of a mammalian eye, is described in U.S. Pat. No. 5,591,720, the contents of which are hereby incorporated by reference. Means of preparing and administering ophthalmic preparations are known in the art (see, e.g., Mullins et al., Chapter 86 In: Remington's Pharmaceutical Sciences, 18th Ed., Gennaro, ed., Mack Publishing Co., Easton, Pa., 1990, pages 1581-1595).

### EXAMPLES

The following examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

### Examples 1

### Compounds

A. Bioactive Agents

The antisense compounds used as exemplary bioactive agents in the studies detailed herein are as follows.

ISIS 2105 is a 2′-deoxyoligonucleotide having a phosphorothioate backbone and the sequence 5′-TTG-CTT-CCA-TCT-TCC-TCG-TC-3′ (SEQ ID NO:11). ISIS 2105 is targeted to the translation initiation codon of the E2 gene of human papillomavirus HPV-11. ISIS 2105 is described in U.S. Pat. No. 5,457,189, hereby incorporated by reference.

ISIS 2503 is a 2′-deoxyoligonucleotide having a phosphorothioate backbone and the sequence 5′-TCC-GTC-ATC-GCT-CCT-CAG-GG-3′ (SEQ ID NO:5). ISIS 2503 is targeted to the translation initiation codon of the human oncogene, Ha-ras. ISIS 2503 is described in U.S. Pat. No. 5,576,208, hereby incorporated by reference.

ISIS 3082 is 2′-deoxyoligonucleotide having a phosphorothioate backbone and the sequence 5′-TGC-ATC-CCC-CAG-GCC-ACC-AT-3′ (SEQ ID NO: 12). ISIS 3082 is targeted to the 3′-untranslated region (3′-UTR) of the murine ICAM-1 gene. ISIS 3082 is described in Stepkowski et al. (J. Immunol., 1994, 153, 5336).

B. Liposomal Components

The lipids used to prepare the liposomal compositions described herein, and commercial sources thereof, are as follows. The emboldened abbreviations for these lipids are used throughout the disclosure.

Chol (cholesterol) is purchased from Avanti Polar Lipids, Inc. (Alabaster, Ala.) or from Sigma Chemical Corp. (St. Louis, Mo.).

PC (phosphatidylcholine) is purchased from Avanti or Sigma.

Egg PC is purchased from Avanti or Sigma.

Soy PC is purchased from Avanti or Sigma.

DLPC (dilauroylphosphatidylcholine) is purchased from Avanti.

DMPC (dimyristoylphosphatidylcholine) is purchased from Avanti or Sigma.

DPPC (dipalmitoylphosphatidylcholine) is purchased from Sigma, Avanti or Genzyme Corp. (Cambridge, Mass.).

DLPG (dilauroylphosphatidylglycerol) is purchased from Avanti or Sigma.

DMPG (dimyristoylphosphatidylglycerol) is purchased from Avanti or Sigma.

DPPG (dipalmitoylphosphatidylglycerol) is purchased from Avanti.

DSPG (distearoylphosphatidylglycerol) is purchased from Avanti.

DPPS (dipalmitoylphosphatidylserine) is purchased from Avanti or Sigma.

DPGS (dipalmitoylglycerosuccinate) is purchased from Avanti.

DSPE-MPEG₂₀₀₀ [N-(carbamoyl-methoxypolyethylene glycol 2000)-1,2-distearoyl-sn-glycero-3-phosphoethanolamine-methoxypoly-ethyleneglycol] is purchased from Avanti.

DOTAP (dioleoyltrimethylammonium propane) is purchased from Avanti.

Card (cardiolipin) is purchased from Avanti.

GM1 (monosialoganglioside G_{M1}) is purchased from Sigma.

### Example 2

### Preparation of Liposomes

A. Preparation of Lipid Film

Lipid stock solutions were prepared at 20 mg/mL in chloroform. For liposomes having DMPG:DPPC:Chol in a molar ratio of 5:57:38, for example, DMPG, DPPC and Chol were dispensed into a 30 mL round bottom flask as follows for 150 μmol of total lipid:

TABLE 1
Lipid Components of 5 Mol % DMPG Liposomes

| | Mole | | | mL stock lipid |
|---|---|---|---|---|
| Component | Ratio | Mole % | mg lipid | solution |
| DMPG | 0.263 | 5 | 5.2 | 0.258 |
| DPPC | 3 | 57 | 62.7 | 3.137 |
| Chol | 2 | 38 | 22.0 | 1.102 |

Chloroform was removed by evaporation using a rotary evaporator, heating at 60 degrees C. with a moderate vacuum. The lipid material dried as a thin film on the flask wall. Evaporation was continued using high vacuum for an additional 30 minutes at 60 degrees C.

B. Lipid Hydration

Oligonucleotide (e.g., ISIS 2105, 2503, 3082 and 3521) was dissolved in water to 100 mg/mL. The solution was made isotonic (80-310 mOsm) with the addition of a small quantity of 5M NaCl as needed. The final solution was filtered through a 0.22 μm membrane. Then, 0.5 mL of the resultant oligo solution was added to the flask containing the lipid film. The flask was rotated at 240 rpm at 60 degrees C. for 5 minutes. The lipid suspension was vortexed heavily to form large multi-lamellar liposomes.

The liposomes were frozen by immersing the flask into a dry ice/acetone bath for 5 minutes. Thawing of the liposomes was accomplished by immersing the flask into a 60 degrees C. water bath as necessary. The preceding freeze/thaw steps were repeated 5 times. The resulting liposome solution appeared “creamy.”

C. Particle Sizing

Large multi-lamellar liposomes were converted into near-uniform unilamellar liposomes by either (1) physical extrusion through polycarbonate membranes (Avestin, Inc., Ottawa, Ontario, Canada) of defined porosity (e.g., 100 nm) or microfluidization with a Model 110 S microfluidizer (Microfluidics International Corp., Newton, Mass.). Either technique produces unilamellar liposomes of approximately 90 to about 110 nm in diameter.

D. Liposome Purification

Nonencapsulated oligonucleotide material was separated from the liposomes by gel permeation chromatography using a Superdex-200 column (Pharmacia Biotech, Inc., Piscataway, N.J.) equilibrated in phosphate-buffered saline, pH 7.4. Encapsulation recovery was typically 25-30% and the final ISIS 2503 concentration in the liposomes was about 7 mg/mL. The liposome fractions were pooled and filter-sterilized through a 0.2 μm membrane (Gelman Sciences, Inc., Ann Arbor, Mich.). Liposomes were stored at 4 degrees C.

### Example 3

### Pharmacokinetics of Liposomes Having Varying Proportions of DMPG

In order to determine the optimal proportion of DMPG for plasma stability in vivo., the following experiments were performed. Liposomes comprising 38 mol % Chol, 1, 10, 15 or 20 mol % DMPG, and (respectively) 61, 52, 47 or 42 mol % DPPC were prepared according to the procedures described in the preceding Examples. The liposomes encapsulated ISIS 2503 as a representative bioactive agent. In order to track the fate of the liposomes, ³H-DPPC (Amersham Life Science, Inc., Arlington Heights, Ill.) was used.

Liposomal formulations were adjusted to 100 mg/mL of total lipid and rats were dosed (1 mL/kg) with radiolabeled liposomes i.v. in the tail vein. Male Sprague Dawley rats (Harlan Sprague Dawley, Inc., Indianapolis, Ind.; n=3 per group) were pre-cannulated for ease of sampling. Plasma was sampled from the rats at various time points after administration for up to 48 hours post-dosing, and radioactivity in the samples was measured by scintillation counting.

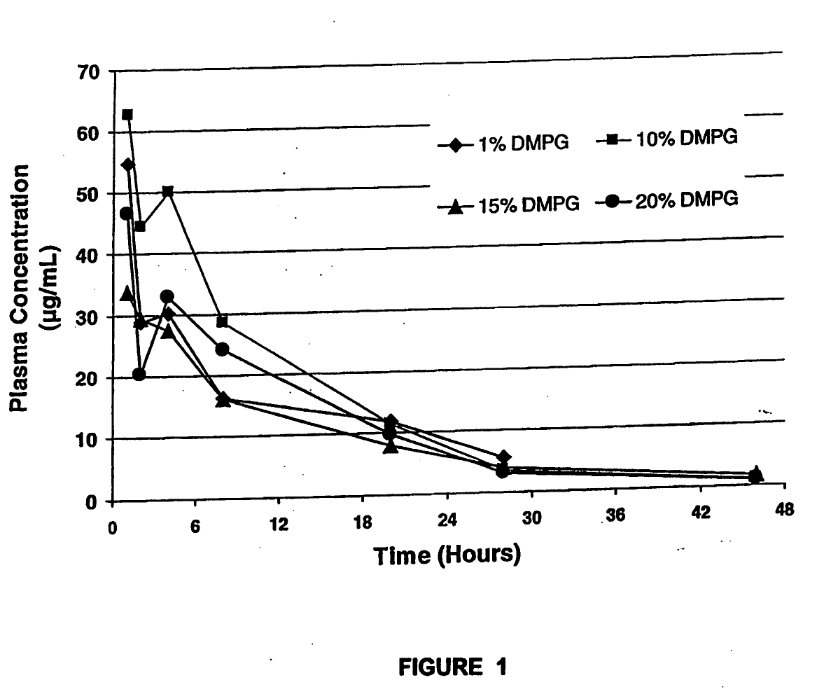

The results of a representative experiment are shown in FIG. 1. Surprisingly, the amount of DMPG in the liposomes can be decreased to as little as about 1 mol % with no depreciable impact on the liposomes stability in plasma in vivo. If anything, liposomes having 1 mol % or 5 mol % DMPG have better stability in plasma than liposomes having greater amounts (i.e., 15 or 20 mol % DMPG).

In sum, encapsulation of phosphorothioate oligonucleotide into DMPG-comprising liposomes greatly modified oligonucleotide pharmacokinetics. ISIS 2503 in such liposomes is cleared slowly from the blood compared with previous experience with unencapsulated oligonucleotide. For example, phosphorothioate oligonucleotide concentration following i.v. infusion of unencapsulated oligonucleotide in monkeys decreases rapidly from circulation with an average distribution half-life of about 1 hour or less (Cossum et al., J. Pharmacol. Exp. Therapeutics, 1993, 267, 1181; Agrawal et al., Clinical Pharmacokinet. 28:7, 1995). In contrast, the half-life of ISIS 2503 in the DMPG-comprising liposome formulation was markedly longer.

### Example 4

### Pharmacokinetics of Liposomes Having Phosphatidylglycerols of Varying Chain Lengths

In order to investigate the effect of modifying the DMPG component of the long circulating liposomes, the following experiments were carried out. Liposomes comprising 38 mol % Chol, 57 mol % DPPC and 5 mol % of one of the “xxPG” lipids listed in Table 2 were prepared. The liposomes contained radiolabeled ³H-DPPC, and were administered to rats, as in the previous Example.

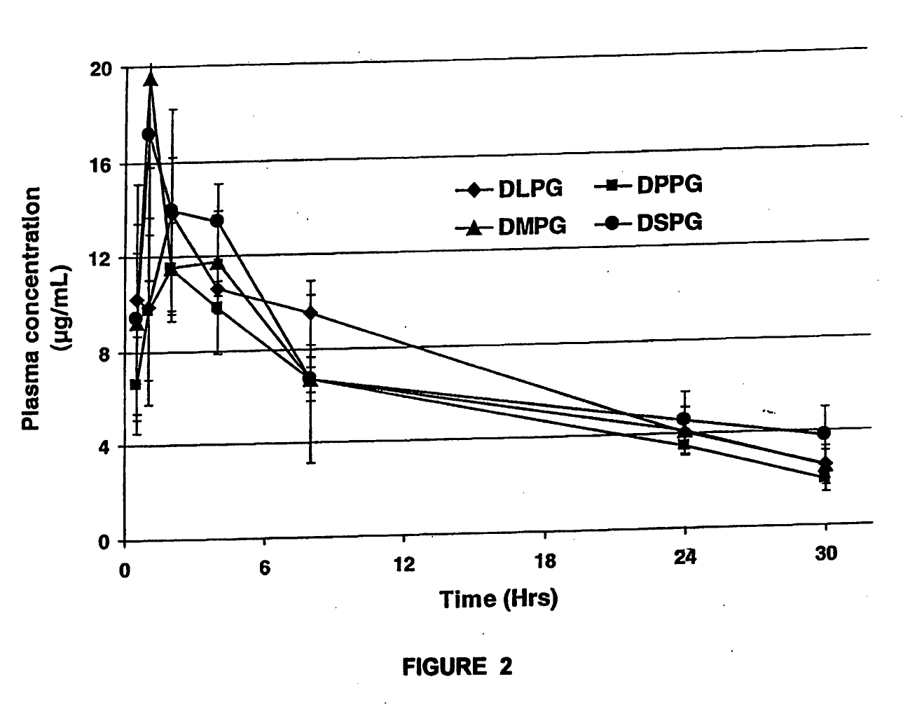

FIG. 2 is a representation of the course of disappearance of radiolabel from the plasma of rats for these liposomal compositions. There was no appreciable difference between the formulations, although the data suggest that liposomes comprising DLPG may provide for a slightly longer period of elevated plasma concentrations relative to the other formulations. In general, phosphatidylglycerol lipids having chains of from about ten to about twenty carbons are preferred for use in the liposomes of the invention, with those having a chain length of from about fourteen to about twenty carbons being more preferred and those with a chain length of from about sixteen to about eighteen carbons being most preferred.

TABLE 2
xxPG (PG = Phosphatidyl Glycerol) Lipids
Used in long-Circulating Liposomes

| Compound | “xx” Group | Chain Length |
|---|---|---|
| DLPG | DL = dilauroyl | 12 carbons |
| DMPG | DM = dimyristoyl | 14 carbons |
| DPPG | DP = dipalmitoyl | 16 carbons |
| DSPG | DS = distearoyl | 18 carbons |

### Example 5

### Pharmacokinetics of Liposomes Having Phosphatidylglycerol-Derived Compounds

In order to investigate the effect of chemical modifications to the DMPG component of the liposomes (other than alterations in the length of the fatty acid chains), the following experiments were carried out with derivatives of phosphatidylglycerol compounds (a.k.a. PG-derived compounds).

In a first formulation, the PG compound was replaced by a lipid having a polar group other the phosphate group. As an example, the glycolipid dipalmitoylglycerosuccinate (DPGS), in which a succinate moiety is present in lieu of a phosphate group, was formulated into the liposomes of the invention in place of DMPG at the same concentration (i.e., 5 mol %). The liposomes, which included ³H-DPPC, encapsulated ISIS 2503, were formulated and tested in rats according to the preceding Examples.

In a second formulation, the phosphate group of the phosphatidylglycerol compound is retained but the dimyristoyl moiety is replaced by a zwitterionic moiety. For example, in the case of dipalmitoylphosphatidylserine (DPPS), the dimyristoyl moiety [—O—CH(OH)—CH₂—CH₂(OH)] attached to the phosphate group of the lipid is replaced by an L-serine moiety [—O—CH₂—CH(NH₃⁺)—COO⁻]. As an example of such liposomes, DPPS was formulated into the liposomes of the invention in place of DMPG at the same concentration (i.e., 5 mol %). The liposomes, which included ³H-DPPC, encapsulated ISIS 2503, were formulated and tested in rats according to the preceding Examples.

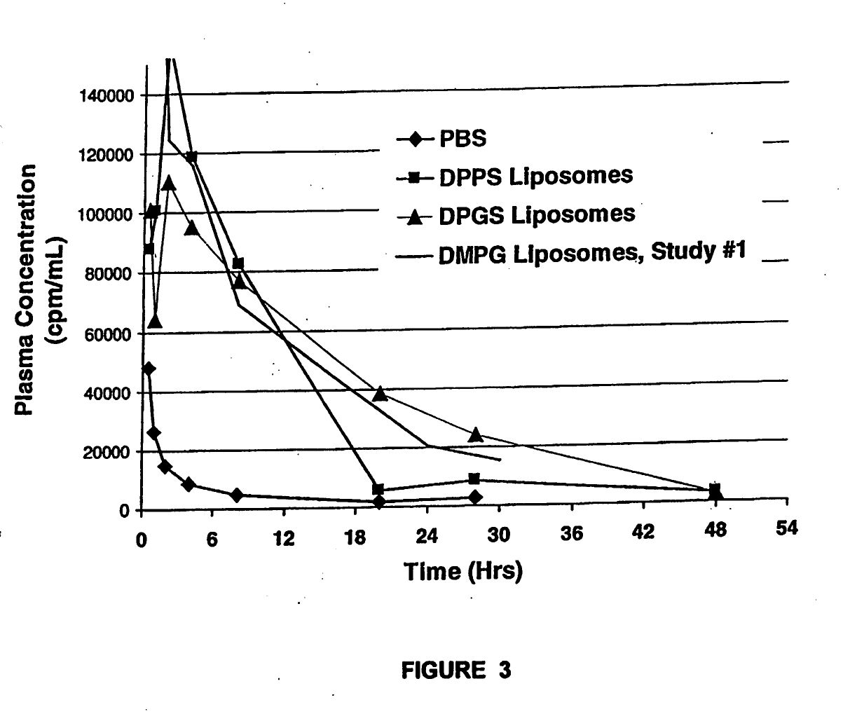

The plasma stabilities of DPGS- and DPPS-comprising liposomes, compared to DMPG-comprising (5 mol %) liposomes, are shown graphically in FIG. 3. Although DPGS seems to be a comparable substitute for DMPG, the plasma stability enhancing effect of DPPS was less readily apparent.

The preceding results are summarized in Table 3, in which the half-lives of various liposomes of the invention (as determined from the data presented in FIGS. 1-3) are given. As can be seen in the upper tier (section I) of Table 3, the optimal mol % of DMPG appears to be around about 1 mol % to about 5 mol %, extending up to about 15 mol %, as these liposomes have plasma half-lives of about 10 to about 14 hours. In contrast, liposomes having 20 mol % DMPG have a slightly less robust plasma stability (half-life of about 9 hours).

As is shown in the middle tier (section II) of Table 3, varying the fatty acid chain length of the PG component of the liposomes did not decrease their plasma stability. Indeed, in the case of DPPG and DSPG (half-lives of 15 and 16 hours, respectively), these changes improve the plasma stability of the liposomes into which they are incorporated.

Finally, the bottom tier (section III) of Table 3 shows that, although DPGS can substitute for DMPG in the long circulating liposomes of the invention (half-life, 12 hours), DPPS confers less plasma stability (half-life, 6 hours). Still, DPPS-comprising liposomes provide more stability to ISIS 2503 than PBS buffer (FIG. 3), indicating that they have some potential as long circulating liposomes.

TABLE 3
Plasma Half-Lives of Liposomes

| Liposome Composition | Plasma Half-Life |
|---|---|
| I. Varying Amounts of DMPG | |
| DMPG - 1 mol % | 12 hours |
| DMPG - 5 mol % | 14 hours |
| DMPG - 10 mol % | 10 hours |
| DMPG - 15 mol % | 12 hours |
| DMPG - 20 mol % | 9 hours |
| II. Varying Fatty Acid Chain Length | |
| DLPG - 5 mol % | 14 hours |
| DMPG - 5 mol % | 14 hours |
| DPPG - 5 mol % | 15 hours |
| DSPG - 5 mol % | 16 hours |
| III. PG-Derived Compounds | |
| DPPS - 5 mol % | 6 hours |
| DPGS - 5 mol % | 12 hours |

### Example 6

### Selective Delivery of Bioactive Agents to Tumors by the Liposomal Compositions of the Invention

One advantage of some sterically stabilized liposomes is their ability to deliver conventional chemotherapeutic agents to tissues, particularly tumors, other than those of the reticuloendothelial system (RES) (Gabizon et al., Proc. Natl. Acad. Sci. USA, 1988, 85, 6949; Papahadjopoulos et al., Proc. Natl. Acad. Sci. USA, 1991, 88, 11460). In disease states where leaky vasculature is characteristic (e.g., tumors, inflammation), prolonging the circulation time via the liposomal oligonucleotide formulations of the invention may allow for more effective delivery of oligonucleotide as well as providing for a less frequent oligonucleotide dosing interval. In order to test the efficacy of liposomal oligonucleotide formulations against tumors, a human-mouse xenograft model was used.

Selective Delivery to Tumors: Liposomes comprising DPPS, DMPG or Chol in the lipid phase, and ISIS 2105 in the aqueous phase, were prepared as in the preceding Examples. ISIS 2105 loaded liposomes comprising 5 mol % of cardiolipin (Card), the monosialoganglioside G_{M1} (GM1) or N-(carbamoyl-methoxypolyethylene glycol 2000)-1,2-distearoyl-sn-glycero-3-phosphoethanolamine-methoxypoly-ethyleneglycol (DSPE-MPEG₂₀₀₀) instead of one of the preceding lipids were prepared in like fashion, except that Card, GM1 or PEG was substituted for, e.g., DMPG at the same final molar concentration. For example, lipid components of DSPE-MPEG₂₀₀₀ liposomes are described in Table 4.

TABLE 4
Lipid Components of 5 Mol %
DSPE-MPEG₂₀₀₀ Liposomes

| | Mole | Mole | mg | mL stock lipid |
|---|---|---|---|---|
| Component | Ratio | % | lipid | solution |
| DSPE-MPEG₂₀₀₀ | 0.263 | 5 | 20.8 | 1.037 |
| DPPC | 3 | 57 | 62.7 | 3.137 |
| Chol | 2 | 38 | 22.0 | 1.102 |

Saline or PBS-buffered formulations of ISIS 2105 were also included as controls in some of the experiments.

Xenografts of human tumor cell lines into BALB/c nude mice were performed essentially as described by Dean et al. (Cancer Res. 56:3499, 1996). Cell lines NCI-H69 and MIA PaCa-2 are available from the American Type Culture Collection (A.T.C.C., Manassas, Va.) as accession numbers ATCC HTB-119 and ATCC CRL-1420, respectively.

Formulations were administered intravenously (i.v.), with H69-bearing mice receiving two 10 mg/kg doses 24 hours apart, and MIA PaCa-bearing mice receiving a single 19.5 mg/kg dose. In either instance, plasma, tumor and other tissue samples were collected post-mortum 24 hours after the final dose, and the concentration of oligonucleotide present in the samples was determined by capillary gel electrophoresis (CGE). The resolving power of CGE allows for the resolution of full-length (“intact”) oligonucleotide from variant synthesis products or those resulting from in vivo degradation.

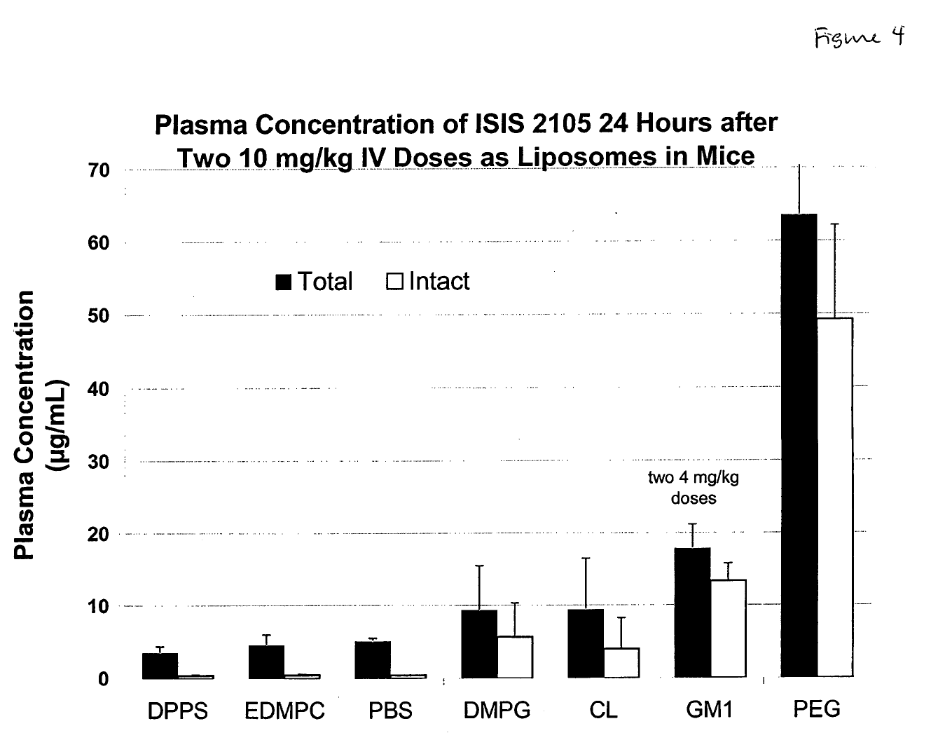

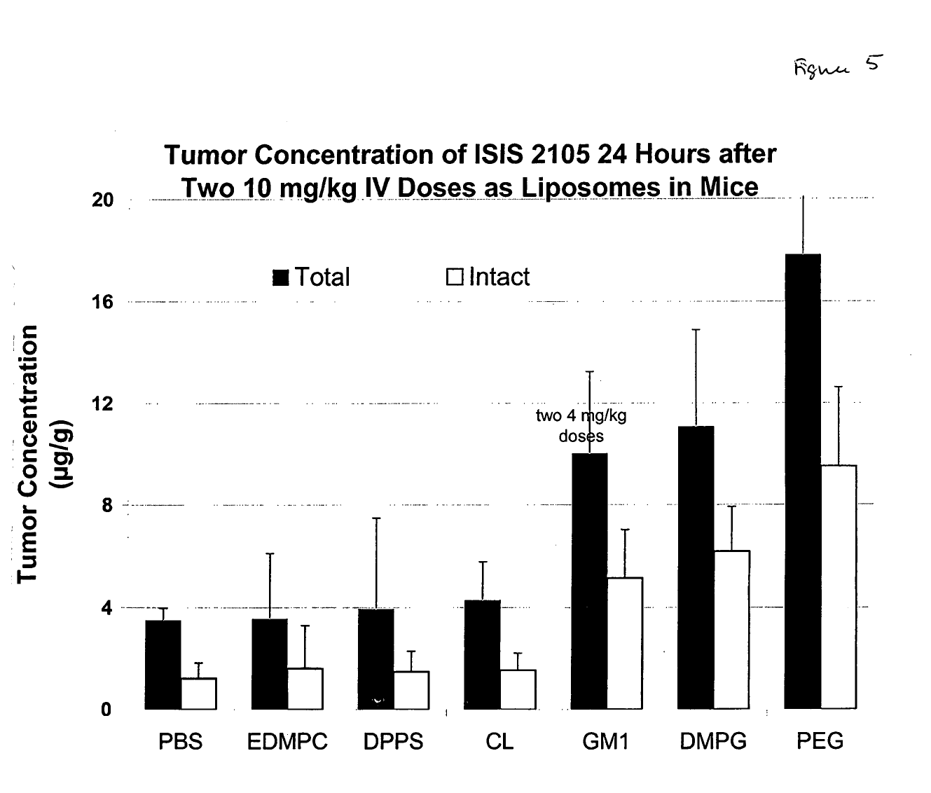

The results for H69 tumors are shown in FIG. 4 (plasma concentrations) and FIG. 5 (tumor concentrations). Unformulated (PBS buffer) of DPPS-liposomally formulated oligonucleotide resulted in the lowest plasma concentrations of bioactive agent 24 hours after dosing, DMPG- and Card-liposomes resulted in medium plasma concentrations, and GM1- and PEG-liposomes resulted in the highest plasma concentrations (FIG. 4). With regard to tumor concentrations, PBS, DPPS- and Card-liposomes all resulted in low concentrations of the bioactive agent in the H69-derived tumors, DMPG-liposomes resulted in moderate tumor concentrations, and GM1- and PEG-liposomes resulted in the highest tumor concentrations (FIG. 5).

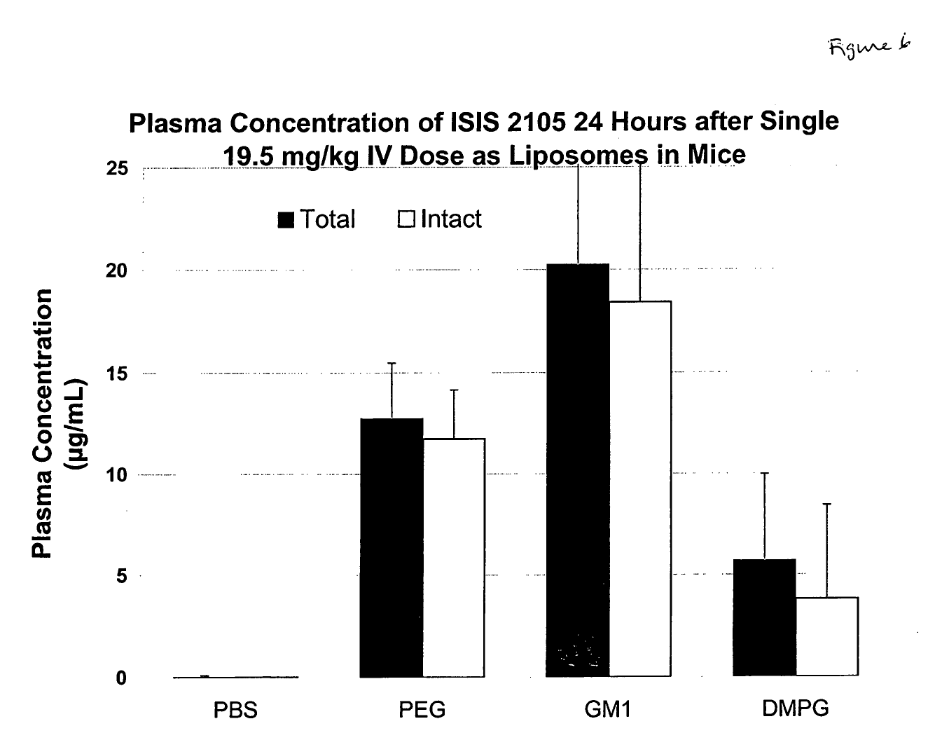

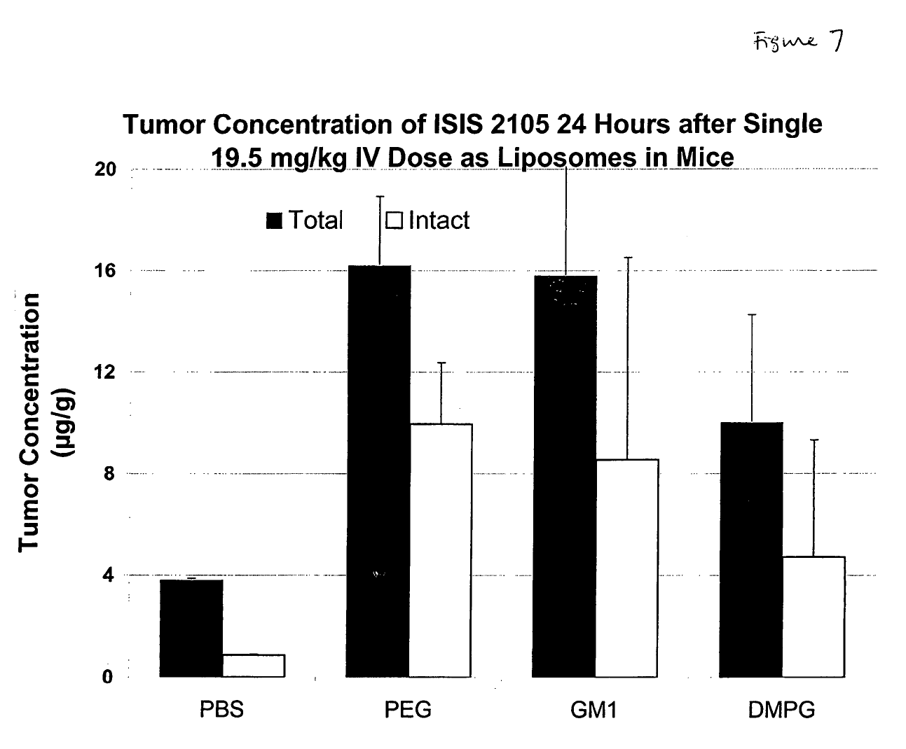

The results for MIA PaCa tumors are shown in FIG. 6 (plasma concentrations) and FIG. 7 (tumor concentrations). Based on the results with H69 tumors, DPPS- and Card-liposomes were not tested for delivery to MIA PaCa tumors. With regard to plasma concentrations, results comparable to those obtained with the H69 studies were obtained, i.e., PBS resulted in low plasma concentrations, DMPG-liposomes resulted in moderate plasma concentrations, and GM1- and PEG-liposomes gave higher plasma concentrations (FIG. 6). With regard to tumor concentrations, PBS resulted in low concentrations of the bioactive agent in the tumors, DMPG-liposomes resulted in moderate tumor concentrations, and GM1- and PEG-liposomes resulted in the tumor concentrations that are somewhat higher than those seen with DMPG-liposomes (FIG. 7).

In summary, GM1 and PEG liposomes had increased plasma circulation stability, and DMPG and cardiolipin liposomes had moderately increased circulation stabilities. However, the DMPG-comprising liposomes selectively delivered ISIS 2105 to tumors to a degree that is disproportionate relative to the extent of plasma stability they confer. For example, DMPG liposomes resulted in plasma concentrations of ISIS 2105 that were less than about ¼th to about ⅙th of those provided by GM1 liposomes (FIG. 4), but delivered at least about 50% of the dose of intact drug delivered by GM1 or PEG liposomes to H69 (FIG. 5) or MIA PaCa (FIG. 7) tumors. DMPG liposomes resulted in an increased accumulation in tumors that is at least about three fold greater than that seen with ISIS 2105 formulated in cardiolipin liposomes or PBS.

Antitumor Activity: The liposomal oligonucleotide formulations of the invention are evaluated for their ability to control the growth of human tumor cells transplanted into BALB/c nude mice. In brief, xenografts of human tumor cell lines in BALB/c nude mice are established as described supra. Liposomal formulations comprising an anticancer agent are administered intraperitoneally (i.p.) or intravenously (i.v.) at a frequency of every other day (q2d) and every third day (q3d). Tumor volume is measured at the indicated times by measuring perpendicular diameters and calculated as described by Dean et al. (Cancer Res. 56:3499, 1996). Enhanced anticancer activity of a bioactive agent due to its encapsulation within a liposome of the invention is indicated by a delayed development of tumors and/or a reduction in their relative size (volume), as well as by decreased mortality of the host animals.

The above results demonstrate that the liposomes of the invention have several advantages over other liposomal formulations. First, the liposomal formulations of the invention result in improved pharmacodynamic properties (e.g., prolonged clearance time from the blood, enhanced biostability in blood and kidney samples, etc.) that result in greater circulating concentrations and stability of full-length oligonucleotides and other bioactive agents. Second, the liposomal formulations of the invention result in enhanced delivery, relative to traditional saline formulations, of the agents encompassed thereby to tumor tissues. Third, due at least in part to the above features, liposomal formulations can achieve higher concentrations and greater specific effects attributable to bioactive agents by using a less frequent dosing regime than used with other formulations. Taken together, these properties are expected to result in an efficacious method for treating an animal, including a human, suffering from a hyperproliferative disease or disorder such as cancer.

### Example 7

### Selective Delivery of Bioactive Agents to Sites of Inflammation by the Liposomal Compositions of the Invention

The present invention provides compositions and methods for modulating the transmigration of leukocytes and thus controlling related undesirable immune responses such as, e.g., inflammation. In order to test the ability of the liposomal compositions of the invention to modulate cellular transmigration, the following experiments were carried out.

The liposomes used in the experiments comprised DMPG:DPPC:Chol (5:57:38 mol %) and encapsulated ISIS 2503. The liposomes were prepared according to the protocols of the preceding Examples. For an animal model of inflammation, the thioglycollate-induced peritonitis model of Watson et al. (Nature, 1991, 349, 164) was used (see also Liao et al., J. Exp. Med., 1997, 185, 1349, and Bogen et al., J. Exp. Med., 1994, 179, 1059).

Groups (n=3 per group) of female C57 Black 6 mice were given unformulated (i.e., in PBS) or liposome-encapsulated, antisense oligonucleotide (ISIS 2503) at an i.v. dose of 6.8 mg/kg 17 hours after thioglycollate exposure. ISIS 2503 was radiolabelled to high specific activity by synthetic incorporation of 35S using hydrogen phosphonate chemistry as described by Stein et al. (Anal. Biochem., 1990, 188, 11). Peritoneal lavages were performed 7 hours after oligonucleotide dosing to collect fluid containing extravasated ISIS 2503, and the radioactivity in the samples was measured by scintillation counting. The percentage of dose present in the peritoneal fluid was calculated according to the formula

$$\frac{Total Radioactivity in Peritoneal Fluid}{Total Radioactivity of Dose} \times 100 \% .$$

The results (Table 5) indicate that the liposomal composition comprising ISIS 2503 delivered over 1% of the total administered dose to the peritoneal fluid in the absence of thioglycollate (“no TG”), whereas administration of ISIS 2503 suspended in PBS resulted in about 0.1% peritoneal delivery. When peritonitis was induced by thioglycollate treatment, the liposomal composition resulted in delivery of about 2.25% of the total dose of ISIS 2503 to the peritoneal fluid. These results demonstrate the ability of the liposomal compositions of the invention to selectively deliver therapeutic agents to sites of inflammation.

TABLE 5
Distribution of ISIS 2503 in Peritoneal Fluid (PF)
of Thioglycollate (TG)-Treated C57BL6 Mice

| | % Dose of ISIS 2503 in PF | |
|---|---|---|
| | when formulated in: | |
| Group | PBS | Liposomes |
| control - no TG | 0.10 | 1.12 |
| peritonitis - w/TG | 0.25 | 2.25 |

### Example 8

### Use of Liposomally Enscapsulated ISIS 3082 of Animal Model for Multiple Sclerosis

Multiple sclerosis (MS) is a slowly progressive demyelinating autoimmune disease (see, generally, The Merck Manual of Diagnosis and Therapy, 15th Ed., pp. 1414-1417, Berkow et al., eds., Rahay, N.J., 1987). Experimental autoimmune encephalomyelitis (EAE) is an artificially induced demyelinating disease that is used as an animal model for MS (for a review, see Burkhardt et al., Rheumatol. Int., 1997, 17, 91). While not wishing to be bound by any particular theory, it was hoped that the liposomes of the invention would increase the degree of extravasation of the bioactive agent encapsulated therein through inflamed sites in the blood-brain barrier (BBB), and would thus be useful for treating MS and other disorders of the central nervous system.

In order to evaluate the ability of the liposomes of the invention to treat EAE, liposomes (DMPG:DPPC:Chol, 5:57:38 mol %) encapsulating ISIS 3082 (1.5 or 3.8 mg/kg) were prepared as in the preceding Examples. ISIS 3082 is targeted to, and inhibits the expression of, the murine gene encoding ICAM-1. Because ICAM-1 mediates cell:cell interactions in a variety of immune system-related contexts, it was hoped that delivery of ISIS 3082 to appropriate sites in vivo might alleviate some or all of the autoimmunologial responses associated with MS.

EAE was induced in female (SJL×BALB/c)F₁ mice (The Jackson Laboratory, Bar Harbor, Me.) at 6 to 8 weeks of age essentially according to the method of Myers et al. (J. Neuroimmunol., 1992, 41, 1). Groups of mice (n=7 unless otherwise noted) were injected s.c. in the hind footpads and the base of the tail with 50 to 100 ug of synthetic peptides corresponding to the primary encephalitogenic determinants (i.e., residues 139-151, HCLGKWLGHPDKF-amide) of proteolipid protein (PLP) for SJL and (SJL×BALB/c)F₁ mice (p13, Tuohy et al., J. Immunol., 1989, 142, 1523; Multiple Peptide Systems, San Diego, Calif.). Antigen was emulsified in complete Freund's adjuvant (CFA, Difco, Detroit, Mich.) and fortified with 4 mg/mL of heat-killed H37Ra Mycobacterium tuberculosis. Mice were injected s.c. on days 0 and 2 and, at the same time, 500 ng of pertussis toxin (Sigma Chemical Co., St. Louis, Mo.) was administered i.v. Animals were watered by hand during periods of paralysis.

The liposomal formulations were administered i.v. daily during disease progression. EAE was clinically assessed according to the procedures and criteria of Myers et al. (J. Immunol., 1993, 151, 2252). In brief, disease severity incidence and time of onset were measured according to defined grades of symptoms, from Grade 0 (no detectable signs of EAE) to Grade 4 (total paralysis/moribund state) and Grade 5 (death). The “Mean Onset Day” of EAE is calculated from the first day that Grade 1 or higher EAE symptoms were noted in a particular mouse (averaged for the group). The “Mean Peak Severity” represents the average within an experimental group of the highest grade of EAE attained by each mouse.

The results demonstrate that the liposomes of the invention encapsulating ISIS 3082 provide relief from the severest symptoms of EAE and delay the onset of the disease. With regard to the severity of the symptoms of the disease, the Mean Peak Severity was 2.6 Grade Units for the control group (n=5), to which empty liposomes were administered, compared to 1.57 Grade Units for the group given liposomally formulated ISIS 3082 at a dose of 3.8 mg/kg. In the case of the group given liposomally formulated ISIS 3082 at a dose of 1.5 mg/kg, the symptoms of EAE were graded as relatively mild during the early course of the disease. Subsequently, however, 3 mice died fairly late in the course of EAE; although the cause of death in these instances was not determined, and may have been due to, e.g., pathogenic infection, the deaths of these animals resulted in them receiving a grade of 5 on the EAE symptom scale. As a result, the Mean Peak Severity of the group receiving liposomally formulated ISIS 3082 at a dose of 3.8 mg/kg was similar to that of the control group (2.5 Grade Units). In any event, however, ISIS 3082 formulated in the liposomes of the invention extended the Mean Onset Day of EAE at either dose (14.7 days, 1.5 mg/kg dose; 16.7 days, 3.8 mg/kg dose) relative to The Mean Onset Day (13.2 days) for the control group.

These results demonstrate that the liposomes of the invention may be used to deliver bioactive agents to sites of inflammation within the central nervous system, and that such liposomes may be used to delay the onset of, and/or reduce the severity of the symptoms of, an autoimmune disease such as MS. These results also demonstrate that inhibition of a cellular adhesion molecule, such as ICAM-1, by a bioactive agent (including, but not limited to, an antisense compound) results in a delay in the onset of, and a reduction in the severity of the symptoms of, autoimmune diseases such as MS.

1. A liposome comprising about 1-25 mol % of a phosphatidylglycerol component and about 38-62 mol % of a phosphatidylcholine component.

2. The liposome of claim 1 comprising about 15-25 mol % of a phosphatidylglycerol component and about 38-47 mol % of a phosphatidylcholine component.

3. The liposome of claim 1 comprising about 5-15 mol % of the phosphatidylglycerol component and about 47-57 mol % of the phosphatidylcholine component.

4. The liposome of claim 1 comprising about 3-10 mol % of the phosphatidylglycerol component and about 52-60 mol % of the phosphatidylcholine component.

5. The liposome of claim 1 comprising about 5 mol % the phosphatidylglycerol component and about 57 mol % of the phosphatidylcholine component.

6. The liposome of claim 1 comprising about 1 mol % of the phosphatidylglycerol component and about 59-62 mol % of the phosphatidylcholine component.

7. The liposome of claim 1 wherein the phosphatidylglycerol component comprises a phosphatidylglycerol having a first fatty acid and a second fatty acid.

8. The liposome of claim 7 wherein a hydrocarbon chain of the first fatty acid comprises 10-20 carbons.

9. The liposome of claim 7 wherein a hydrocarbon chain of the first fatty acid comprises 14-20 carbons.

10. The liposome of claim 7 wherein a hydrocarbon chain of the first fatty acid comprises 16-18 carbons.

11. The liposome of claim 7 wherein a hydrocarbon chain of the second fatty acid comprises 10-20 carbons.

12. The liposome of claim 7 wherein a hydrocarbon chain of the second fatty acid comprises 14-20 carbons.

13. The liposome of claim 7 wherein a hydrocarbon chain of the second fatty acid comprises 16-18 carbons.

14. The liposome of claim 7 wherein a hydrocarbon chain of the first fatty acid comprises a different number of carbons than a hydrocarbon chain of the second fatty acid.

15. The liposome of claim 7 wherein a hydrocarbon chain of the first fatty acid comprises the same number of carbons as a hydrocarbon chain of the second fatty acid.

16. The liposome of claim 1 wherein the phosphatidylglycerol component comprises a dilauroylphosphatidylglycerol, a dimyristoylphosphatidylglycerol, a dipalmitoylphosphatidylglycerol, a disteroylphosphatidylglycerol, phosphatidylglycerol-derived compound or a mixture thereof.

17. The liposome of claim 1 wherein the phosphatidylglycerol component comprises a dimyristoylphosphatidylglycerol.

18. The liposome of claim 16 wherein the phosphatidylglycerol-derived compound is a glycolipid.

19. The liposome of claim 18 wherein the glycolipid is dipalmitoylglycerosuccinate.

20. The liposome of claim 16 wherein the phosphatidylglycerol-derived compound is a dipalmitoylphosphatidylserine.

21. The liposome of claim 1 wherein the phosphatidylcholine component comprises a phosphatidylcholine having a first fatty acid and a second fatty acid.

22. The liposome of claim 21 wherein a hydrocarbon chain of the first fatty acid comprises 10-20 carbons.

23. The liposome of claim 21 wherein a hydrocarbon chain of the first fatty acid comprises 14-20 carbons.

24. The liposome of claim 21 wherein a hydrocarbon chain of the first fatty acid comprises 16-18 carbons.

25. The liposome of claim 21 wherein a hydrocarbon chain of the second fatty acid comprises 10-20 carbons.

26. The liposome of claim 21 wherein a hydrocarbon chain of the second fatty acid comprises 14-20 carbons.

27. The liposome of claim 21 wherein a hydrocarbon chain of the second fatty acid comprises 16-18 carbons.

28. The liposome of claim 21 wherein a hydrocarbon chain of the first fatty acid comprises a different number of carbons than a hydrocarbon chain of the second fatty acid.

29. The liposome of claim 21 wherein a hydrocarbon chain of the first fatty acid comprises the same number of carbons as a hydrocarbon chain of the second fatty acid.

30. The liposome of claim 21 wherein the phosphatidylcholine component comprises an egg phosphatidylcholine, a soybean phosphatidylcholine, a dilauroylphosphatidylcholine, a dipalmitoylphosphatidylcholine or a mixture thereof

31. The liposome of claim 21 wherein the phosphatidylcholine component comprises a dipalmitoylphosphatidylcholine.

32. The liposome of claim 1 wherein the phosphatidylglycerol component comprises a dimyristoylphosphatidylglycerol, and the phosphatidylcholine component comprises a dipalmitoylphosphatidylcholine.

33. The liposome of claim 1 wherein the phosphatidylglycerol component is about 5 mol % and the phosphatidylcholine component is about 57 mol %, and wherein the phosphatidylglycerol component comprises a dimyristoylphosphatidylglycerol, and the phosphatidylcholine component comprises a dipalmitoylphosphatidylcholine.

34. The liposome of any of claims 1-33 further comprising a sterol component.

35. The liposome of claim 34 wherein the sterol component comprises a cholesterol.

36. The liposome of claim 34 comprising about 33-42 mol % of the sterol component.

37. The liposome of claim 34 comprising about 38 mol % of the sterol component.

38. The liposome of claim 34 comprising is about 5 mol % of the phosphatidylglycerol component, about 57 mol % of the phosphatidylcholine component and about 38 mol % of the sterol component, and wherein the phosphatidylglycerol component comprises a dimyristoylphosphatidylglycerol, the phosphatidylcholine component comprises a dipalmitoylphosphatidylcholine and the sterol component comprises a cholesterol.

39. The liposome of claim 1 encapsulating a bioactive agent.

40. The liposome of claim 39 wherein the bioactive agent is an anticancer agent.

41. The liposome of claim 39 wherein the bioactive agent is an anti-inflammatory agent.

42. The liposome of claim 39 wherein the bioactive agent is an oligonucleotide.

43. The liposome of claim 42, wherein the oligonucleotide is an siRNA, a hemimer, a molecular decoy, an aptamer, or an antisense oligomer.

44. A pharmaceutical composition comprising the liposome of claim 1 or 33.

45. A method of preventing cancer or reducing the rate of growth of a tumor or cancer in a mammal, comprising administering to said mammal a prophylactically or therapeutically effective amount of the liposome of claim 40.

46. A method of preventing or reducing the severity of inflammation in a mammal, comprising administering to said mammal a prophylactically or therapeutically effective amount of the liposome of claim 41.

47. A liposome of any of claims 1-33 wherein the liposome encapsulates a ribonucleoprotein complex.

48. A liposome of claim 34 wherein the liposome encapsulates a ribonucleoprotein complex.
